(19) United States
(12) Patent Application Publication
(10) Pub. No.: US 2026/0321388 A1
(43) Pub. Date: Oct. 15, 2026

(54) SYSTEMS AND METHODS FOR DETERMINING A USER INTERFACE TYPE

(71) Applicant: ResMed Digital Health Inc., San Diego, CA (US)

(72) Inventors: Gregory Robert Peake, Sydney (AU); Andrew Chan, Sydney (AU); Sakeena De Souza, Sydney (AU); Priyanshu Gupta, Hornsby, NSW (AU); Albert Jack Greenwood Woffenden, Sydney (AU); Nathan Zersee Liu, Sydney (AU); Andrew William Gillett, Sydney (AU); Siew Ting Ooi, Singapore (SG); Genevieve Claire Atkinson, Sydney (AU)

(21) Appl. No.: 19/104,144

(22) PCT Filed: Aug. 18, 2023
(86) PCT No.: PCT/US2023/030645

§ 371 Date: Feb. 14, 2025

Related U.S. Application Data
(60) Provisional application No. 63/399,441, filed on Aug. 19, 2022.
(60) Provisional application No. 63/382,067, filed on Nov. 2, 2022.

(51) Int. Cl. G16H 20/40

(52) U.S. Cl. CPC G16H 20/40

(57) ABSTRACT

A method for determining a user interface type comprises receiving data associated with an individual who uses a respiratory therapy system. The method further comprises receiving data associated with a plurality of other individuals. Each of the plurality of other individuals is a user of a respiratory therapy system. The method further comprises, based at least in part on the received data, determining a recommended type of user interface to be worn by the individual during use of the respiratory therapy system.

### CROSS-REFERENCE TO RELATED APPLICATIONS

This application claims the benefit of, and priority to, U.S. Provisional Patent Application No. 63/399,441 filed on Aug. 19, 2022, and U.S. Provisional Patent Application No. 63/382,067 filed on Nov. 2, 2022, each of which is hereby incorporated by reference herein in its entirety.

### TECHNICAL FIELD

The present disclosure relates generally to systems and methods for determining suitable devices and components for a user, and more particularly, to systems and methods for determining suitable devices and components for a user to use during a sleep session based on devices or components used by similar individuals, sensor data, community-driven inputs, or any combinations thereof.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. The pressurized air is delivered via at least a conduit coupled to a respiratory therapy device of the respiratory therapy system, and a user interface that is worn by the individual. However, some individuals find these respiratory therapy systems to be uncomfortable, difficult to use, expensive, aesthetically unappealing, and/or fail to perceive the benefits associated with using the respiratory therapy system. Further, when selecting the appropriate therapy system or its components, individuals often have little guidance on which device and/or components would provide the most positive user experience (e.g., desired or an improved level of comfort or performance), and/or which device user interface best fits their head and/or face and ensures effective delivery of the pressurized air. Moreover, even when selection guidance is available, such as product reviews, the reviews usually only discuss a limited number of product models and are not customized to the individual's needs. Thus, individuals will often find that their personal experience with the reviewed device and component differ from that of the reviewer. As a result, some individuals will elect not to use the respiratory therapy system or discontinue use of the respiratory therapy system absent a demonstration of the severity of their symptoms when respiratory therapy treatment is not used or encouragement or affirmation that the respiratory therapy system is improving their sleep quality and reducing the symptoms of these disorders. Thus, new systems and methods are needed for selecting the respiratory therapy system and any components of the respiratory therapy system, including the user interface to be worn by the individual. The present disclosure is directed to solving these and other problems.

### SUMMARY

According to some implementations of the present disclosure, a method for generating recommendations related to use of a respiratory therapy system includes receiving data associated with an individual who uses a respiratory therapy system. The method also includes receiving data associated with a plurality of other individuals. Each of the plurality of other individuals is a user of a respective respiratory therapy system. The method also includes determining, based at least in part on the received data, a recommended type of user interface to be worn by the individual during use of the respiratory therapy system by the individual.

According to some implementations of the present disclosure, a method for generating recommendations related to use of respiratory therapy system includes receiving data from at least one of a plurality of sensors and at least one of a plurality of community-driven inputs. The data from the plurality of sensors is objectively determined and the data from the plurality of community-driven inputs is subjectively determined. The method further includes determining a measurement score that ranks a plurality of user interfaces based on the data being correlated to user information associated with a first user, each of the user interfaces having a respective score rank. The method further includes identifying one or more suitable user interfaces from the plurality of user interfaces based on the respective score rank of each of the user interfaces. The method further includes providing a recommendation for the first user to use at least one of the one or more suitable user interfaces.

According to some implementations of the present disclosure, a system for generating recommendations related to use of a respiratory therapy system comprises a control system and a memory. The memory stores machine-readable instructions. The control system includes one or more processors configured to execute the machine-readable instructions to implement a method. The method includes receiving data associated with an individual who uses a respiratory therapy system. The method also includes receiving data associated with a plurality of other individuals. Each of the plurality of other individuals is a user of a respective respiratory therapy system. The method also includes determining, based at least in part on the received data, a recommended type of user interface to be worn by the individual during use of the respiratory therapy system by the individual.

A system for generating recommendations related to use of a respiratory therapy system includes a database and a control system. The database is configured to store user information associated with a first user of the respiratory therapy system. The control system is communicatively coupled to the database, and is configured to implement a method. The method includes receiving, by the control system, data from at least one of a plurality of sensors and a plurality of social media channels. The data from the plurality of sensors is objectively determined and the data from the plurality of social media channels is subjectively determined by one or more of a social media influencer and a key opinion leader. The method further includes determining, by the control system, a measurement score that ranks a plurality of user interfaces based on the data being correlated to the user information associated with the first user, each of the user interfaces having a respective score rank. The method further includes identifying, by the control system, one or more suitable user interfaces from the plurality of user interfaces based on the respective score rank of each of the user interfaces. The method further includes providing, by the control system, a recommendation for the first user to use at least one of the one or more suitable user interfaces.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3A is a perspective view of a respiratory therapy device of the system of FIG. 1, according to some implementations of the present disclosure;

FIG. 3B is a perspective view of the respiratory therapy device of FIG. 3A illustrating an interior of a housing, according to some implementations of the present disclosure;

FIG. 4A is a perspective view of a user interface, according to some implementations of the present disclosure;

FIG. 4B is an exploded view of the user interface of FIG. 4A, according to some implementations of the present disclosure;

FIG. 5A is a perspective view of a user interface, according to some implementations of the present disclosure;

FIG. 5B is an exploded view of the user interface of FIG. 5A, according to some implementations of the present disclosure;

FIG. 6A is a perspective view of a user interface, according to some implementations of the present disclosure;

FIG. 6B is an exploded view of the user interface of FIG. 6A, according to some implementations of the present disclosure;

FIG. 7 illustrates an exemplary timeline for a sleep session, according to some implementations of the present disclosure;

FIG. 8 illustrates an exemplary hypnogram associated with the sleep session of FIG. 3, according to some implementations of the present disclosure;

FIG. 9 is a flow diagram of a method for determining a user interface type, according to some implementations of the present disclosure;

FIG. 10 is a flow diagram of a method for a data and community-driven recommendation engine for sleep-related and/or respiratory-related devices and components, according to some implementations of the present disclosure; and

FIG. 11 depicts a user device presenting a recommended device and/or component for use with a respiratory therapy system, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale and are provided merely to illustrate the instant disclosure. Several aspects of the disclosure are described below with reference to example applications for illustration.

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate, and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea. As will be understood, a sleep session as described herein can alternatively be referred to as a therapy session, during which an individual may receive respiratory therapy, or can comprise or consist of a therapy session.

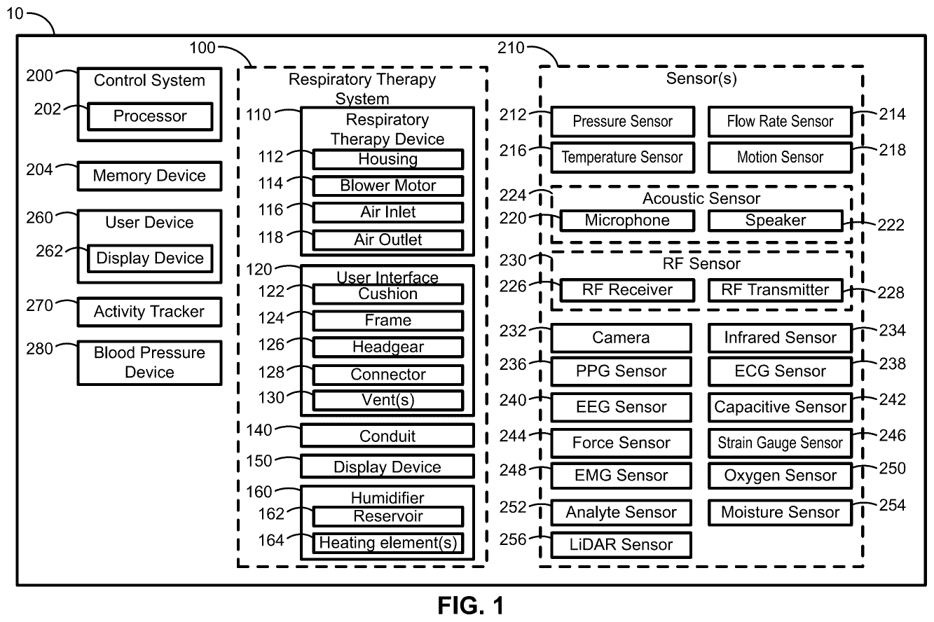

Referring to FIG. 1, a system 10, according to some implementations of the present disclosure, is illustrated. The system 10 can include a respiratory therapy system 100, a control system 200, a memory device 204, and one or more sensors 210. The system 10 may additionally or alternatively include a user device 260, an activity tracker 270, and a blood pressure device 280. The system 10 can be used to analyze data from a plurality of individuals and determine a type of user interface to be worn by a target individual during use of the respiratory therapy system 100.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

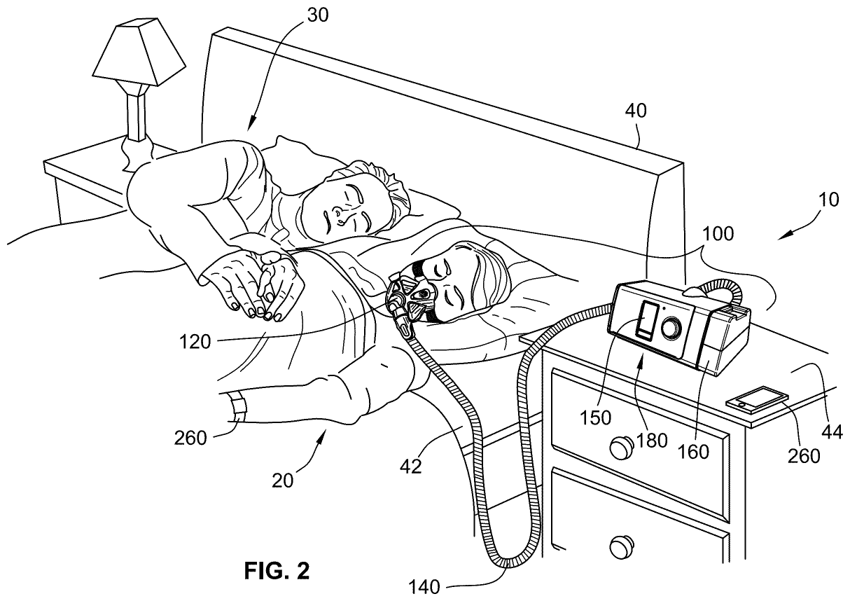

As shown in FIG. 2, the respiratory therapy system 100 can be used to treat a user 20. In this example, the user 20 of the respiratory therapy system 100 and a bed partner 30 are in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

Referring back to FIG. 1, the respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about −20 L/min and about 150 L/min, while maintaining a positive pressure (relative to the ambient pressure).

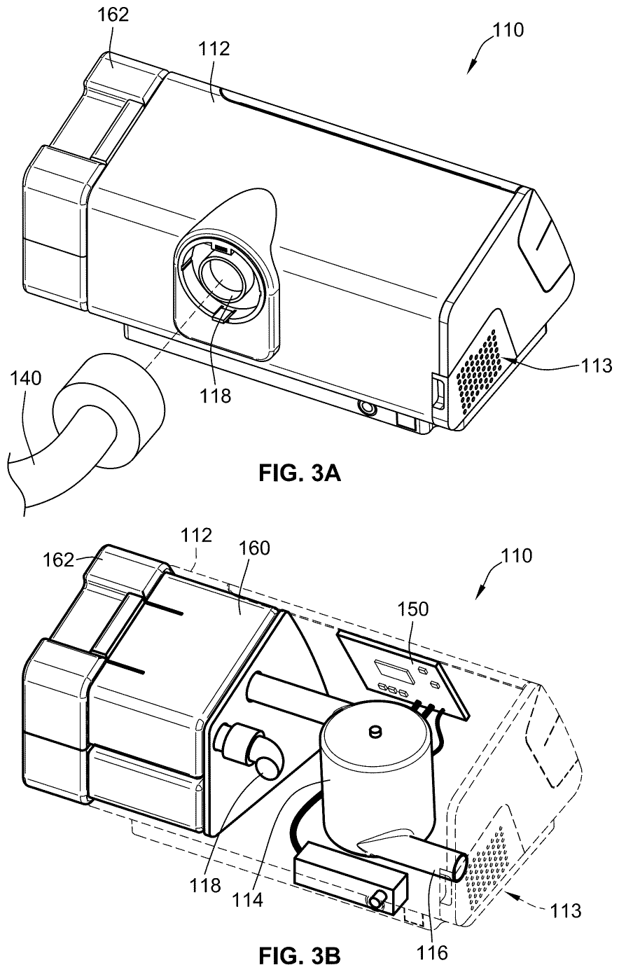

The respiratory therapy device 110 includes a housing 112, a blower motor 114, an air inlet 116, and an air outlet 118. Referring to FIGS. 3A and 3B, the blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). As shown in FIGS. 3A and 3B, the housing 112 can also include a vent 113 to allow air to pass through the housing 112 to the air inlet 116. As described below, the conduit 140 is coupled to the air outlet 118 of the respiratory therapy device 110.

Referring back to FIG. 1, the user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The one or more vents 130 can be used for permitting the escape of carbon dioxide and other gases exhaled by the user 20. The user interface 120 generally can include any suitable number of vents (e.g., one, two, five, ten, etc.).

As shown in FIG. 2, in some implementations, the user interface 120 is a facial mask (e.g., a full-face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

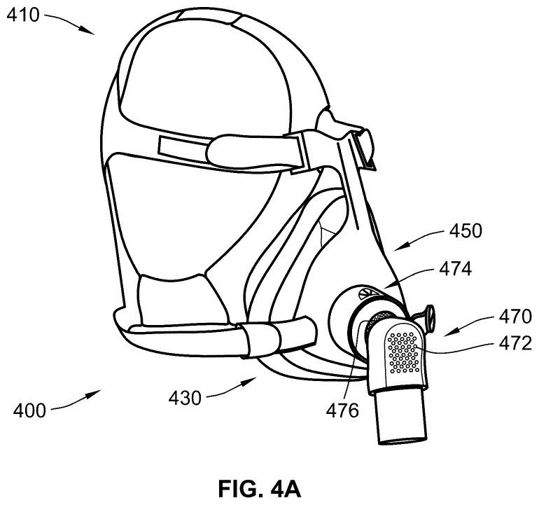

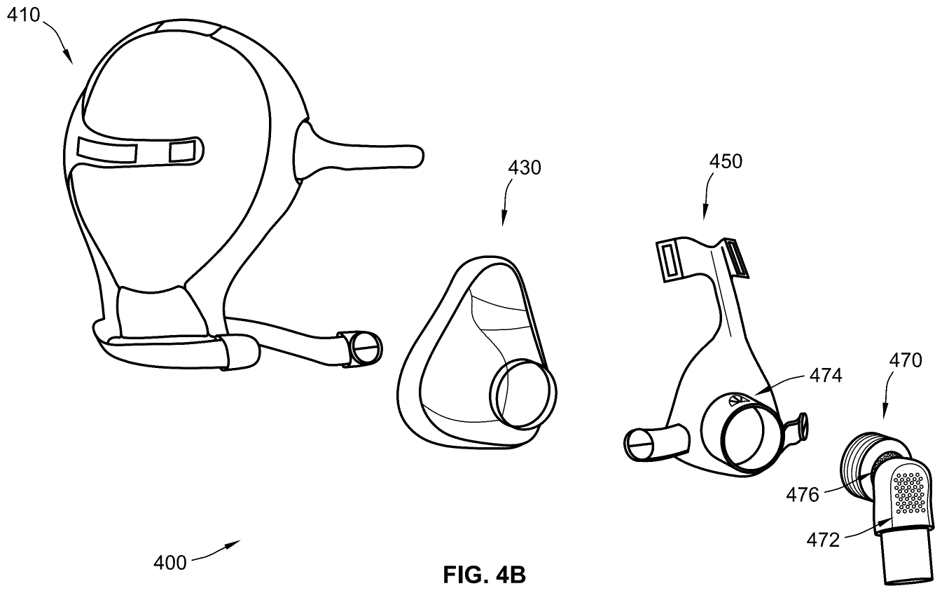

Referring to FIGS. 4A and 4B, a user interface 400 that is the same as, or similar to, the user interface 120 (FIG. 1) according to some implementations of the present disclosure is illustrated. The user interface 400 generally includes a cushion 430 and a frame 450 that define a volume of space around the mouth and/or nose of the user. When in use, the volume of space receives pressurized air for passage into the user's airways. In some implementations, the cushion 430 and frame 450 of the user interface 400 form a unitary component of the user interface. The user interface 400 can also include a headgear 410, which generally includes a strap assembly and optionally a connector 470. The headgear 410 is configured to be positioned generally about at least a portion of a user's head when the user wears the user interface 400. The headgear 410 can be coupled to the frame 450 and positioned on the user's head such that the user's head is positioned between the headgear 410 and the frame 450. The cushion 430 is positioned between the user's face and the frame 450 to form a seal on the user's face. The connector 470 is configured to couple to the frame 450 and/or cushion 430 at one end and to a conduit of a respiratory therapy device (not shown). The pressurized air can flow directly from the conduit of the respiratory therapy system into the volume of space defined by the cushion 430 (or cushion 430 and frame 450) of the user interface 400 through the connector 470). From the user interface 400, the pressurized air reaches the user's airway through the user's mouth, nose, or both. Alternatively, where the user interface 400 does not include the connector 470, the conduit of the respiratory therapy system can connect directly to the cushion 430 and/or the frame 450.

In some implementations, the connector 470 may include one or more vents 472 (e.g., a plurality of vents) located on the main body of the connector 470 itself and/or one or a plurality of vents 476 (“diffuser vents”) in proximity to the frame 450, for permitting the escape of carbon dioxide (CO₂) and other gases exhaled by the user. In some implementations, one or a plurality of vents, such as vents 472 and/or 476 may be located in the user interface 400, such as in frame 450, and/or in the conduit 140. In some implementations, the frame 450 includes at least one anti-asphyxia valve (AAV) 474, which allows CO₂ and other gases exhaled by the user to escape in the event that the vents (e.g., the vents 472 or 476) fail when the respiratory therapy device is active. In general, AAVs (e.g., the AAV 474) are present for full-face masks (e.g., as a safety feature); however, the diffuser vents and vents located on the mask or connector (usually an array of orifices in the mask material itself or a mesh made of some sort of fabric, in many cases replaceable) are not necessarily both present (e.g., some masks might have only the diffuser vents such as the plurality of vents 476, other masks might have only the plurality of vents 472 on the connector itself).

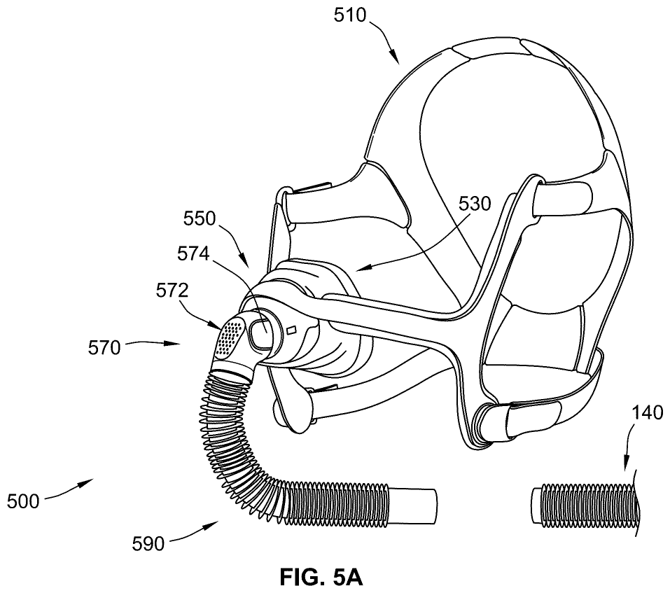

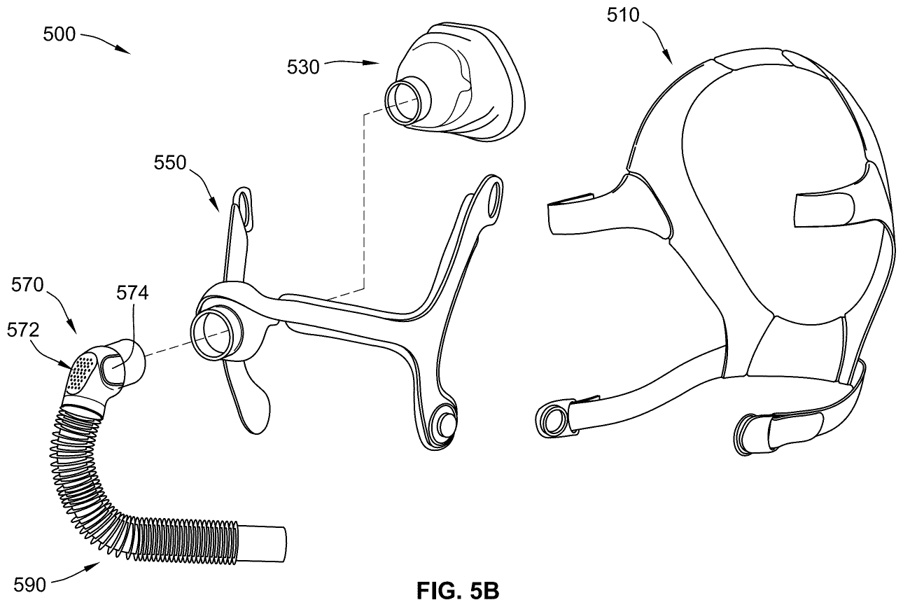

Referring to FIGS. 5A and 5B, a user interface 500 that the is the same, or similar to, the user interface 120 (FIG. 1) according to some implementations of the present disclosure is illustrated. The user interface 500 differs from the user interface 400 (FIGS. 4A and 4B) in that the user interface 500 is an indirect user interface, whereas the user interface 400 is a direct user interface. The user interface 500 includes a headgear 510 (e.g., as a strap assembly), a cushion 530, a frame 550, a connector 570, and a user interface conduit 590 (often referred to as a minitube or a flexitube). The user interface 500 is an indirectly connected user interface because pressurized air is delivered from the conduit 140 of the respiratory therapy system to the cushion 530 and/or frame 550 through the user interface conduit 590, rather than directly from the conduit 140 of the respiratory therapy system.

In some implementations, the cushion 530 and frame 550 form a unitary component of the user interface 500. Generally, the user interface conduit 590 is more flexible than the conduit 140 of the respiratory therapy system 100 (FIG. 1) described above and/or has a diameter smaller than the diameter of the than the than the conduit 140. The user interface conduit 590 is typically shorter that conduit 140. Similar to the headgear 310 of user interface 300 (FIGS. 3A-3B), the headgear 510 of user interface 500 is configured to be positioned generally about at least a portion of a user's head when the user wears the user interface 500. The headgear 510 can be coupled to the frame 550 and positioned on the user's head such that the user's head is positioned between the headgear 510 and the frame 550. The cushion 530 is positioned between the user's face and the frame 550 to form a seal on the user's face. The connector 570 is configured to couple to the frame 550 and/or cushion 530 at one end and to the conduit 590 of the user interface 500 at the other end. In other implementations, the conduit 590 may connect directly to frame 550 and/or cushion 530. The conduit 590, at the opposite end relative to the frame 550 and cushion 530, is configured to connect to the conduit 140. The pressurized air can flow from the conduit 140 of the respiratory therapy system, through the user interface conduit 590, and the connector 570, and into a volume of space define by the cushion 530 (or cushion 530 and frame 550) of the user interface 500 against a user's face. From the volume of space, the pressurized air reaches the user's airway through the user's mouth, nose, or both.

In some implementations, the connector 570 includes a plurality of vents 572 for permitting the escape of carbon dioxide (CO₂) and other gases exhaled by the user when the respiratory therapy device is active. In such implementations, each of the plurality of vents 572 is an opening that may be angled relative to the thickness of the connector wall through which the opening is formed. The angled openings can reduce noise of the CO₂ and other gases escaping to the atmosphere. Because of the reduced noise, acoustic signal associated with the plurality of vents 572 may be more apparent to an internal microphone, as opposed to an external microphone. Thus, an internal microphone may be located within, or otherwise physically integrated with, the respiratory therapy system and in acoustic communication with the flow of air which, in operation, is generated by the flow generator of the respiratory therapy device and passes through the conduit and to the user interface 500.

In some implementations, the connector 570 optionally includes at least one valve 574 for permitting the escape of CO₂ and other gases exhaled by the user when the respiratory therapy device is inactive. In some implementations, the valve 574 (an example of an anti-asphyxia valve) includes a silicone (or other suitable material) flap that is a failsafe component, which allows CO₂ and other gases exhaled by the user to escape in the event that the vents 572 fail when the respiratory therapy device is active. In such implementations, when the silicone flap is open, the valve opening is much greater than each vent opening, and therefore less likely to be blocked by occlusion materials.

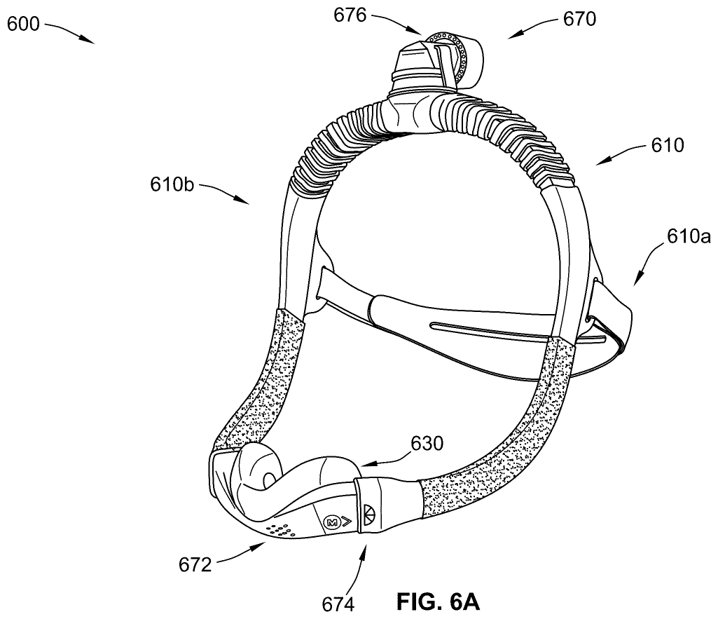

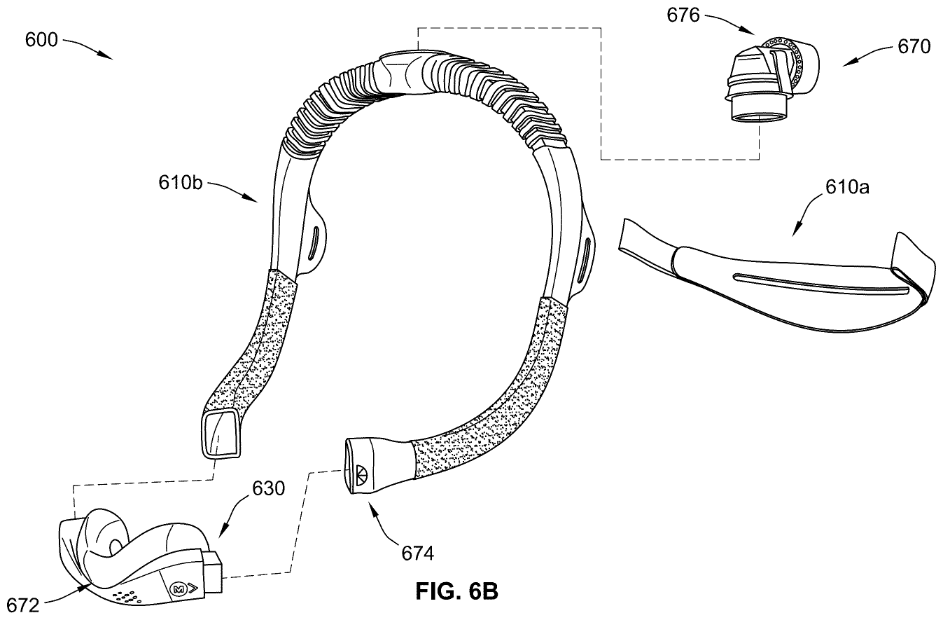

Referring to FIGS. 6A and 6B, a user interface 600 that is the same as, or similar to, the user interface 120 (FIG. 1) according to some implementations of the present disclosure is illustrated. The user interface 600 is similar to the user interface 500 in that it is an indirect user interface. The indirect headgear user interface 600 includes headgear 610, a cushion 630, and a connector 670. The headgear 610 includes strap 610a and a headgear conduit 610B. Similar to the user interface 400 (FIGS. 4A-4B) and user interface 500 (FIGS. 5A-5B), the headgear 610 is configured to be positioned generally about at least a portion of a user's head when the user wears the user interface 600. The headgear 610 includes a strap 610a that can be coupled to the headgear conduit 610B and positioned on the user's head such that the user's head is positioned between the strap 610a and the headgear conduit 610B. The cushion 630 is positioned between the user's face and the headgear conduit 610B to form a seal on the user's face.

The connector 670 is configured to couple to the headgear 610 at one end and a conduit of the respiratory therapy system at the other end (e.g., conduit 140). In other implementations, the connector 670 is not included and the headgear 610 can alternatively connect directly to conduit of the respiratory therapy system. The headgear conduit 610B can be configured to deliver pressurized air from the conduit of the respiratory therapy system to the cushion 630, or more specifically, to the volume of space around the mouth and/or nose of the user and enclosed by the user cushion. The headgear conduit 610B is hollow to provide a passageway for the pressurized air. Both sides of the headgear conduit 610B can be hollow to provide two passageways for the pressurized air. Alternatively, only one side of the headgear conduit 610B can be hollow to provide a single passageway. In the implementation illustrated in FIGS. 6A and 6B, headgear conduit 610B comprises two passageways which, in use, are positioned at either side of a user's head/face. Alternatively, only one passageway of the headgear conduit 610B can be hollow to provide a single passageway. The pressurized air can flow from the conduit of the respiratory therapy system, through the connector 670 and the headgear conduit 610B, and into the volume of space between the cushion 630 and the user's face. From the volume of space between the cushion 630 and the user's face, the pressurized air reaches the user's airway through the user's mouth, nose, or both.

In some implementations, the cushion 630 includes a plurality of vents 672 on the cushion 630 itself. Additionally or alternatively, in some implementations, the connector 670 includes a plurality of vents 676 (“diffuser vents”) in proximity to the headgear 610, for permitting the escape of carbon dioxide (CO₂) and other gases exhaled by the user when the respiratory therapy device is active. In some implementations, the headgear 610 may include at least one plus anti-asphyxia valve (AAV) 674 in proximity to the cushion 630, which allows CO₂ and other gases exhaled by the user to escape in the event that the vents (e.g., the vents 672 or 676) fail when the respiratory therapy device is active.

Referring back to FIG. 1, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

Referring to FIG. 3A, the conduit 140 includes a first end 142 that is coupled to the air outlet 118 of the respiratory therapy device 110. The first end 142 can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end 142 can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a my Air™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, as shown in FIGS. 3A and 3B, air flows from the air inlet 116 through the blower motor 114, and then through the humidifier 160 before exiting the respiratory therapy device 110 via the air outlet 118.

Referring back to FIG. 1, while the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid-state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct). Thus, while the control system 200 and the memory device 204 are shown as independent components in the block diagram of FIG. 1, they may be components of some other component of the system 10, such as the user device 260, the respiratory therapy device 110, etc.

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

The one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared (IR) sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a Light Detection and Ranging (LiDAR) sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the IR sensor 234, the PPG sensor 236, the ECG sensor 238, the EEG sensor 240, the capacitive sensor 242, the force sensor 244, the strain gauge sensor 246, the EMG sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a user interface leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleep stage, an apnea-hypopnea index (AHI), pressure settings of the respiratory therapy device 110, or any combination thereof. The one or more events can include snoring, apneas, central apneas, obstructive apneas, mixed apneas, hypopneas, a user interface leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or user interface leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20 (FIG. 2), a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 can comprise an acoustic sensor (such as the acoustic sensor 224 discussed herein) and/or an RF sensor (such as the RF sensor 230 discussed herein), which can generate motion data as further discussed herein. In such implementations, the motion sensor 218, the acoustic sensor, and/or the RF sensor can be disposed in a portable device, such as the user device 260. Further, while FIG. 1 and FIG. 2 show the respiratory therapy device 110 as including its own display device 150, in some implementations the respiratory therapy device 110 may not include its own display device, as is discussed herein. In some implementations, the motion sensor 218 alternatively or additionally generates one or more signals representing bodily movement of the user, from which may be obtained a signal representing a sleep state of the user, for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. The microphone 220 can be coupled to or integrated in a wearable device, such as a smartwatch, smart glasses, earphones or earbuds, or other head-wearable devices. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones.

The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260, and/or can be coupled to or integrated in a wearable device, such as a smartwatch, smart glasses, earphones or ear buds, or other head-wearable devices.

The microphone 220 and the speaker 222 can be used as separate devices. In some implementations, the microphone 220 and the speaker 222 can be combined into an acoustic sensor 224 (e.g., a sonar sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30 (FIG. 2). Based at least in part on the data from the microphone 220 and/or the speaker 222, the control system 200 can determine a location of the user 20 and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g., a radar sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user, to determine air flow of the mouth and/or nose of the user, to determine a time when the user enters the bed 40 (FIG. 2), and to determine a time when the user exits the bed. In some implementations, the camera 232 includes a wide-angle lens or a fisheye lens.

The IR sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120, in the associated headgear (e.g., straps, etc.), in a head band or other head-worn sensor device, etc.

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 252 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 252 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The LiDAR sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 10, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 214 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, a microphone, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a user interface leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 includes a display device 262. The user device 260 can be, for example, a mobile device such as a smartphone, a tablet computer, a gaming console, a smartwatch, a laptop computer, or the like. In some implementations, the user device 260 is a portable device, such as a smartphone, a tablet computer, a smartwatch, a laptop computer, etc. Alternatively, the user device 260 can be an external sensing system, a television (e.g., a smart television), or another smart home device (e.g., a smart speaker(s) such as Google Home, Amazon Echo, Amazon Alexa, etc.). In some implementations, the user device is a wearable device (e.g., a smartwatch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 10 also includes the activity tracker 270. The activity tracker 270 is generally used to aid in generating physiological data associated with the user. The activity tracker 270 can include one or more of the sensors 210 described herein, such as, for example, the motion sensor 218 (e.g., one or more accelerometers and/or gyroscopes), the PPG sensor 236, and/or the ECG sensor 238. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 10 also includes the blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp, chirp, triangle, sinewave, etc.), other schemes such as PSK, FSK etc., pulsed continuous wave, and/or spread in ultra-wideband ranges (which may include spreading, PRN codes or impulse systems).

While the control system 200 and the memory device 204 are described and shown in FIG. 1 as being a separate and distinct component of the system 10, in some implementations, the control system 200 and/or the memory device 204 are integrated in the user device 260 and/or the respiratory therapy device 110. Thus, the control system 200 and/or the memory device 204 can be disposed within the housing 112 of the respiratory therapy device 110. Alternatively, in some implementations, the control system 200 or a portion thereof (e.g., the processor 202) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 10 is shown as including all the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

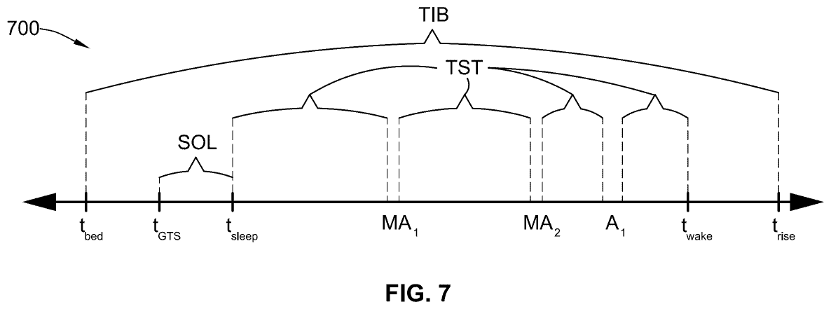

Referring now to FIG. 7, as used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user has laid or sat down in the bed (or another area or object on which they intend to sleep) and has turned on the respiratory therapy device 110 and donned the user interface 120. The sleep session can thus include time periods (i) when the user is using the respiratory therapy system 100, but before the user attempts to fall asleep (for example when the user lays in the bed reading a book); (ii) when the user begins trying to fall asleep but is still awake; (iii) when the user is in a light sleep (also referred to as stage 1 and stage 2 of non-rapid eye movement (NREM) sleep); (iv) when the user is in a deep sleep (also referred to as slow-wave sleep, SWS, or stage 3 of NREM sleep); (v) when the user is in rapid eye movement (REM) sleep; (vi) when the user is periodically awake between light sleep, deep sleep, or REM sleep; or (vii) when the user wakes up and does not fall back asleep. The sleep session may also be referred to as a therapy session, or may comprise a therapy session, which can be understood to be the period of time within the sleep session during which the individual is engaged in respiratory therapy (e.g., the use of a respiratory therapy system).

The sleep session is generally defined as ending once the user removes the user interface 120, turns off the respiratory therapy device 110, and gets out of bed. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user, ending when the respiratory therapy device 110 stops supplying the pressurized air to the airway of the user, and including some or all the time points in between, when the user is asleep or awake.

FIG. 7 illustrates an exemplary timeline 700 for a sleep session. The timeline 700 includes an enter bed time (t_{bed}), a go-to-sleep time (t_{GTS}), an initial sleep time (tₛₗₑₑₚ), a first micro-awakening MA₁, a second micro-awakening MA₂, an awakening A, a wake-up time (t_{wake}), and a rising time (tᵣᵢₛₑ).

The enter bed time t_{bed} is associated with the time that the user initially enters the bed (e.g., bed 40 in FIG. 2) prior to falling asleep (e.g., when the user lies down or sits in the bed). The enter bed time t_{bed} can be identified based at least in part on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based at least in part on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based at least in part on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based at least in part on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based at least in part on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}), or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based at least in part on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, as shown in the timeline 700, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

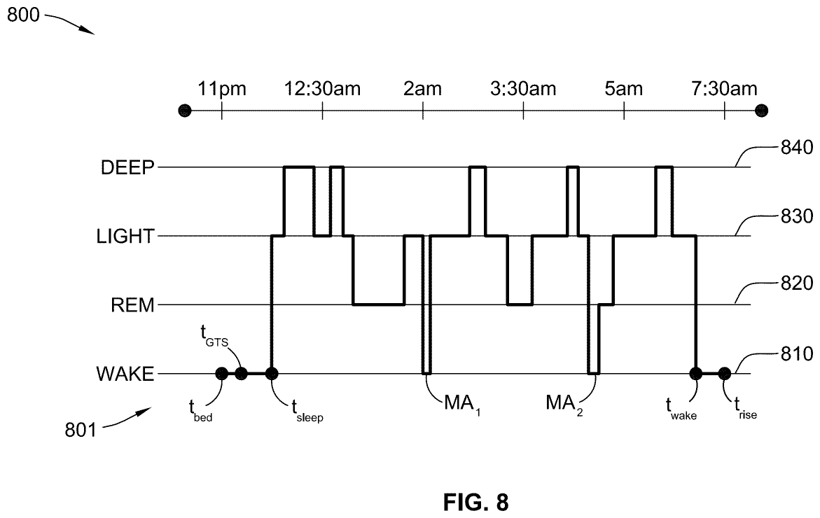

Referring to FIG. 8, an exemplary hypnogram 800 corresponding to the timeline 700 of FIG. 7, according to some implementations, is illustrated. As shown, the hypnogram 800 includes a sleep-wake signal 801, a wakefulness stage axis 810, a REM stage axis 820, a light sleep stage axis 830, and a deep sleep stage axis 840. The intersection between the sleep-wake signal 801 and one of the axes 810-840 is indicative of the sleep stage at any given time during the sleep session.

The sleep-wake signal 801 can be generated based at least in part on physiological data associated with the user (e.g., generated by one or more of the sensors 210 described herein). The sleep-wake signal can be indicative of one or more sleep stages, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 800 is shown in FIG. 8 as including the light sleep stage axis 830 and the deep sleep stage axis 840, in some implementations, the hypnogram 800 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration amplitude ratio, an inspiration-expiration duration ratio, a number of events per hour, a pattern of events, or any combination thereof. Information describing the sleep-wake signal can be stored in the memory device 204.

The hypnogram 800 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 8), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based at least in part on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and ₁₀).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based at least in part on, for example, data generated by the motion sensor 218, the microphone 220, the camera 232, or any combination thereof. The go-to-sleep time can be determined based at least in part on, for example, data from the motion sensor 218 (e.g., data indicative of no movement by the user), data from the camera 232 (e.g., data indicative of no movement by the user and/or that the user has turned off the lights), data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

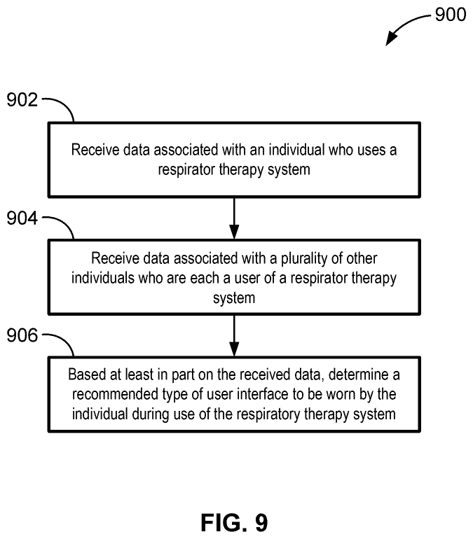

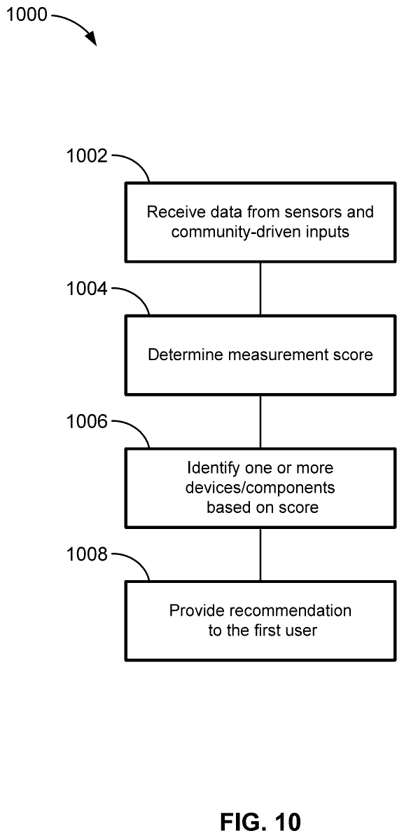

Referring to FIGS. 9 and 10, methods and systems are described for generating recommendations related to use of a respiratory therapy system by an individual or a user. These methods and systems as described herein can provide individuals with guidance on which sleep-related and/or respiratory-related therapy devices and components would provide the most positive user experience (e.g., desired or improved level of comfort or performance), thereby leading to enhanced therapy outcomes. Moreover, these methods and systems and in providing selection guidance that can be customized to the individual's needs. As used herein, the term “one or more sleep-related and/or respiratory-related therapy devices and components” generally includes any device or component that the user may use during a sleep session related to their sleep and/or their use of a respiratory therapy system, including a user interface, a conduit, a respiratory therapy device, another component of the respiratory therapy system, components or devices that are not part of the respiratory therapy system (e.g., a mouthguard, a mandibular reposition device, a humidifier, a white noise machine, etc.), and the like. In generally, any device and/or component that is recommended using any of the features disclosed with respect to FIGS. 9 and/or 10, and/or any other feature disclosed herein, can include any device and/or component that the user may use that is related to a sleep session and/or the user's use of a respiratory therapy system, including a user interface, a conduit, a respiratory therapy device, another component of the respiratory therapy system, components or devices that are not part of the respiratory therapy system (e.g., a mouthguard, a mandibular reposition device, a humidifier, a white noise machine, etc.), and the like.

The terms “individual” and “user” are both used herein to describe the person that is using a respiratory therapy system, and are generally interchangeable with each other. These methods and systems can generate a recommended type of user interface for an individual to wear during use of a respiratory therapy system, and/or a recommended component for use with the respiratory therapy system. These methods and systems may be used in combination with or separately from any of the features described herein with respect to FIGS. 1-8.

FIG. 9 illustrates a method 900 for generating recommendations for a user interface and/or other component for use with a respiratory therapy system based on a comparison between the individual who uses the respiratory therapy system and other similar individuals who also use a respiratory therapy system.

Step 902 of method 900 includes receiving data associated with an individual who uses a respiratory therapy system (such as the respiratory therapy system 100 of the system 10). Step 904 of method 900 includes receiving data associated with a plurality of other individuals. Each of the plurality of individuals is a user of a respective respiratory therapy system (such as the respiratory therapy system 100 of the system 10). Step 906 of method 900 includes determining, based at least in part on the received data, a recommended type of user interface to be worn by the individual during use of the respiratory therapy system by the individual.

The data associated with the individual can include data generated by one or more sensors, such as any of the sensors 210 of the system 10 in FIG. 1. The sensors can include one or more of a pressure sensor, a flow rate sensor, a temperature sensor, a motion sensor, a microphone, a speaker, a radio-frequency (RF) receiver, a RF transmitter, a camera, an infrared sensor, a photo-plethysmogram (PPG) sensor, an electrocardiogram (ECG) sensor, an electroencephalography (EEG) sensor, a capacitive sensor, a force sensor, a strain gauge sensor, an electromyography (EMG) sensor, an oxygen sensor, an analyte sensor, a moisture sensor, a LiDAR sensor, a facial scanner, a blood oxygen level sensor, a blood pressure sensor, a heart rate sensor, a photodetector, an air pressure sensor, a device usage tracker, or any combination thereof. Other types of sensors can include sensors linked with various types of user devices capable of collecting user information, such as a smartphone, a laptop or desktop computer, a personal digital assistant (PDA), a tablet computer, an automotive infotainment system, a smart mirror, a television, a respiratory therapy device (e.g., positive airway pressure (PAP) devices or non-PAP alternative treatment devices, such as a mandibular advancement appliance, positional therapy device, oral muscle training tool), a sleep enhancement or therapy device, a nearable device, a wearable monitoring device (e.g., a smartwatch or heart-rate monitor), an airable device, digital calendars, etc.

In some implementations, the data generated by the one or more sensors can include (but is not limited to) one of the following: facial scan data, audio data, user interface usage data, user interface leakage data, and user interface noise data, physiological data, a sleep diagnosis (e.g., of insomnia, restless leg syndrome, parasomnia, narcolepsy, circadian rhythm sleep-wake disorders, sleep apnea, etc.), a sleep schedule, a duration of sleep, restlessness, severity of sleep disorder, user device usage, use of a type of sleep enhancement device (e.g., sleep enhancement headband), etc. In some implementations, the sensor generated data can also include respiratory information. The respiratory information can include any suitable categories. For example, the respiratory information can include use of a respiratory therapy system, a type of respiratory therapy device, a duration of respiratory therapy usage (e.g., how many hours per night a user uses their respiratory therapy system), a length of respiratory therapy usage (e.g., for what period of time, such as days, months, and/or years, a user has been using their respiratory therapy system), a sleep schedule, a duration of sleep, medical conditions, an apnea hypopnea index, a sleep score, etc.

In some implementations, the recommended type of user interface is selected from a plurality of types of user interface. In some implementations, the recommended type of user interface is a type of user interface of the plurality of types of user interface that minimizes an amount of unintentional air leak when worn by the individual during use of the respiratory therapy system. In some implementations, the recommended type of user interface is a type of user interface of the plurality of types of user interface that is predicted to be most comfortable for the individual. In some implementations, a comfort of each of the plurality of types of user interfaces is based at least in part on a rating given to each of the plurality of types of user interfaces by one or more of the plurality of other individuals.

In some implementations, method 900 further comprises determining a type of user interface worn by the individual during one or more prior uses of the respiratory therapy system. In response to the type of user interface previously worn by the individual being different than the recommended type of user interface, method 900 further comprises sending a recommendation to the individual to switch to the recommended type of user interface during one or more future uses of the respiratory therapy system. In some implementations, method 900 further comprises determining a type of user interface worn by the individual during one or more prior uses of the respiratory therapy system. In response to the type of user interface previously worn by the individual being identical to the recommended type of user interface, method 900 further comprises sending a recommendation to the individual to continue using the recommended type of user interface during one or more future uses of the respiratory therapy system. In some implementations, determining the type of user interface worn by the individual during the one or more prior uses of the respiratory therapy system includes (i) receiving input from the individual indicative of the type of user interface worn by the individual during the one or more prior uses of the respiratory therapy system, (ii) analyzing data associated with the one or more prior uses of the respiratory therapy system to determine the type of user interface worn by the individual during the one or more prior uses of the respiratory therapy system, or (iii) both (i) and (ii).

In some implementations, the data for each respective other individual of the plurality of other individuals includes (i) data associated with a user interface worn by the respective other individual and (ii) personal data associated with the respective other individual.

In some implementations, the personal data associated with the respective other individual is indicative of one or more physical characteristics of the respective other individual, an ambient temperature of a location of the respective other individual during use of the respective respiratory therapy system, an ambient humidity of the location of the respective other individual during use of the respective other respiratory therapy system, sleeping habits of the respective other individual, a severity of a respiratory condition of the respective other individual, a typical sleeping position of the respective other individual, an amount of movement of the respective other individual during one or more uses of the respective respiratory therapy system, a breathing path of the respective other individual during one or more uses of the respective respiratory therapy system, an ethnicity of the respective other individual, or any combination thereof.

In some implementations, the one or more physical characteristics of the respective other individual includes a size of a face of the respective other individual, a shape of the face of the respective other individual, a size of a mouth of the respective other individual, a shape of the mouth of the respective other individual, a size of a nose of the respective other individual, a shape of a nose of the respective other individual, a presence of facial hair on the respective other individual, one or more skin characteristics of the respective other individual, or any combination thereof.

In some implementations, the respiratory condition of the respective other individual is obstructive sleep apnea (OSA), and the severity of the OSA is based on an apnea-hypopnea index (AHI) of the respective other individual. In some implementations, the typical sleeping position of the respective other individual is (i) the respective other individual lying on a back of the respective other individual or (ii) the respective other individual lying on a side of the respective other individual. In some implementations, the breathing path of the respective other individual is through a mouth of the respective other individual, through a nose of the respective other individual, or through both the mouth and the nose of the respective other individual. In some implementations, the data associated with the user interface worn by the respective other individual is indicative of a type of the user interface, a tightness of the user interface, a sleep position of the respective other individual during the use of the respective respiratory therapy system, a rating of the user interface given by the respective other individual, an amount of unintentional air leak out of the user interface worn by the respective other individual, or any combination thereof. In some implementations, the tightness of the user interface (also referred to as the level of tightness or the amount of tightness) is quantified using a numerical scale. In some implementations, a first end of the numerical scale indicates that the user interface is under-tightened, and a second end of the numerical scale indicates that the user interface is over-tightened. In some implementations, the data associated with the user interface is indicative of one or more skin abnormalities of the individual following one or more sleep sessions, and the tightness of the user interface is determined from the one or more skin abnormalities. In some implementations, the one or more skin abnormalities includes one or more marks on a face of the individual caused by the user interface, a discoloration on the face of the individual caused by the user interface, or both.

In some implementations, method 900 further comprises identifying, based at least in part on the received data, one or more of the plurality of other individuals that match the individual. Determining the user interface type to be worn by the individual can be based at least in part on the identification of the one or more of the plurality of other individuals that match the individual. In some implementations, the matching one or more individuals share at least one of the one or more physical characteristics with the individual. In some implementations, the matching one or more individuals have an identical severity of the respiratory condition as the individual, and identical typical sleeping position as the individual, an identical amount of movement during one or more uses of the respective respiratory therapy system as the individual, an identical breathing path during one or more uses of the respective respiratory therapy system as the individual, or any combination thereof. In some implementations, the method 900 further comprises identifying, based at least in part on the received data, one or more of the plurality of other individuals that match the individual. The method 900 further comprises selecting the type of user interfaced worn by at least one of the matching other individuals as the type of user interface to be worn by the individual during use of the respiratory therapy system.

In some implementations identifying the matching one or more other individuals includes identifying one or more of the plurality of other individuals who have (i) a similar nose as the individual, (ii) a similar mouth as the individual, (iii) a similar cheekbone height as the individual, (iv) a similar face width as the individual, (v) a similar amount of facial hair as the individual, or (v) any one of (i) through (iv). In some implementations the matching one or more other individuals with the similar nose as the individual have a similar nose size as the individual, a similar nose shape as the individual, a similar nose height as the individual, a similar nose width as the individual, a similar nose depth as the individual, or any combination thereof. In some implementations, the matching one or more other individuals with the similar mouth as the individual have a similar mouth size as the individual, a similar mouth shape as the individual, a similar mouth height as the individual, a similar mouth width as the individual, or any combination thereof.

In some implementations, the method 900 further comprises, in response to determining that the matching one or more other individuals use a first type of user interface, selecting the first type of user interface as the type of user interface to be worn by the individual during the use of the respiratory therapy system. In some implementations, the method 900 further comprises, in response to determining that the matching one or more other individual do not use a first type of user interface, selecting a second type of user interface as the type of user interface to be worn by the individual during the use of the respiratory therapy system. In some implementations, the matching one or more other individuals have a similar nose as the individual. In some implementations, the matching one or more other individuals have an identical breathing path (e.g., breathing through the mouth, breathing through the nose, etc.). In some implementations, the first type of user interface is a nasal pillow mask or a nasal mask, and the second type of user interface is a full-face mask. In some implementations, the nasal pillow mask and the nasal mask do not cover a mouth of the individual, and the full-face mask does cover the mouth of the individual.

In some implementations, the method 900 further comprises, based at least in part on the data associated with the plurality of other individuals, providing a recommendation to the individual associated with a fit of the recommended type of user interface. In some implementations, the recommendation associated with the fit of the recommended type of user interface includes a recommendation to adjust a tightness of the recommended type of user interface, a recommendation to use a specified level of tightness for the recommended type of user interface, a recommendation to adjust a size of the recommended type of user interface, a recommendation to use a specified size for the recommended type of user interface, a recommendation to adjust a position of the recommended type of user interface on a face of the individual, a recommendation to use a specified position of the recommended type of user interface on the face or the individual, or any combination thereof.

In some implementations, identifying the matching one or more of the plurality of other individuals includes determining an ambient environment of the individual during the use of the respiratory therapy system by the individual, and identifying one or more of the plurality of other individuals who have an ambient environment during the use of the respective respiratory therapy system that matches the ambient environment of the individual. In some implementations, identifying the matching one or more of the plurality of other individuals includes determining one or more physical characteristics of the individual, determining one or more physical characteristics of each of the plurality of other individuals, and identifying one or more of the plurality of other individuals who share at least one physical characteristic with the individual. In some implementations, the one or more physical characteristics includes a size of a nose, a shape of a nose, a size of a mouth, a shape of a mouth, a height of cheekbones, a width of a face, a presence of facial hair, one or more skin characteristics, or any combination thereof.

In some implementations, the data associated with the individual includes data associated with a face scan of the individual performed after one or more sleep sessions. In some implementations, the method 900 further comprises identifying one or more of the plurality of other individuals having a face scan that matches the face scan of the individual, and selecting the type of user interface worn by at least one of the other individuals having a matching face scan as the type of user interface to be worn by the individual during user of the respiratory therapy system. In some implementations, the data associated with the face scan of the individual includes data reproducible as one or more images of a face of the individual, data reproducible as one or more videos of the face of the individual, data associated with one or more anatomical characteristics of the face of the individual, data associated with one or more depth measurements of the face of the individual, or any combination thereof. In some implementations, the data associated with the one or more anatomical characteristics of the face of the individual includes data associated with a height of a nose of the individual, a width of the nose of the individual, a depth of the nose of the individual, a height of a mouth of the individual, a width of the mouth of the individual, a distance between the nose of the individual and a mouth of the individual, or any combination thereof.

In some implementations, at least a portion of the data associated with the face scan of the individual is generated with a smartphone of the individual. In some implementations, the data associated with each respective other individual of the plurality of other individuals includes data associated with a face scan of the respective other individual.

In some implementations, the method 900 further comprises identifying, based at least in part on the data associated with the face scan of the individual and the data associated with the face scan of the plurality of other individuals, one or more of the plurality of other individuals that match the individual, and selecting the type of user interface worn by at least one of the matching other individuals as the type of user interface to be worn by the individual during use of the respiratory therapy system. In some implementations, the method 900 further comprises estimating an unintentional leak of the recommended type of user interface when worn by the individual during use of the respiratory therapy system, based at least in part on the face scan of the individual. In some implementations, estimating the unintentional leak is further based at least in part on the face scan of the plurality of other individuals, a scan of the recommended type of user interface, or both.

In some implementations, the method 900 further comprises determining a value of one or more sleep metrics of the individual, determining a value of the one or more sleep metrics for at least one of the plurality of other individuals, and taking an action based at least in part on the determined values. In some implementations, the action includes transmitting to the individual a recommendation to use a specific type of user interface, a recommendation to change the type of user interface being used, a recommendation to adjust a fit of the user interface being worn, a recommendation to adjust one or more settings on the respiratory therapy system of the individual, a recommendation to use a personalized therapy algorithm with the respiratory therapy system of the individual, or any combination thereof.

In some implementations, the method 900 further comprises inputting the received data into a trained machine learning algorithm and receiving from the trained machine learning algorithm the type of user interface to be worn by the individual during use of the respiratory therapy system.

FIG. 10 illustrates a method 1000 for generating recommendations for a user interface and/or other component for use with a respiratory therapy system based on data received from community-driven inputs. This recommendation can be delivered to a user of the respiratory therapy system, who is referred to herein as the first user. At step 1002, data is received from a plurality of sensors and from community-driven inputs. The data from the plurality of sensors is objectively determined and the data from the community-driven inputs is subjectively determined.

Similar to method 900, the plurality of sensors can include any of the sensors 210 of the system 10 in FIG. 1, including a pressure sensor, a flow rate sensor, a temperature sensor, a motion sensor, a microphone, a speaker, a radio-frequency (RF) receiver, a RF transmitter, a camera, an infrared sensor, a photo-plethysmogram (PPG) sensor, an electrocardiogram (ECG) sensor, an electroencephalography (EEG) sensor, a capacitive sensor, a force sensor, a strain gauge sensor, an electromyography (EMG) sensor, an oxygen sensor, an analyte sensor, a moisture sensor, a LiDAR sensor, a facial scanner, a blood oxygen level sensor, a blood pressure sensor, a heart rate sensor, a photodetector, an air pressure sensor, a device usage tracker, or any combination thereof. Other types of sensors can include sensors linked with various types of user devices capable of collecting user information associated with the first user, such as a smartphone, a laptop or desktop computer, a personal digital assistant (PDA), a tablet computer, an automotive infotainment system, a smart mirror, a television, a respiratory therapy device (e.g., positive airway pressure (PAP) devices or non-PAP alternative treatment devices, such as a mandibular advancement appliance, positional therapy device, oral muscle training tool), a sleep enhancement or therapy device, a nearable device, a wearable monitoring device (e.g., a smartwatch or heart-rate monitor), an airable device, digital calendars, etc.

Also similar to method 900, the data generated by the one or more sensors can include (but is not limited to) one of the following: facial scan data, audio data, user interface usage data, user interface leakage data, and user interface noise data, physiological data, a sleep diagnosis (e.g., of insomnia, restless leg syndrome, parasomnia, narcolepsy, circadian rhythm sleep-wake disorders, sleep apnea, etc.), a sleep schedule, a duration of sleep, restlessness, severity of sleep disorder, user device usage, use of a type of sleep enhancement device (e.g., sleep enhancement headband), etc. In some implementations, the sensor generated data can also include respiratory information. The respiratory information can include any suitable categories. For example, the respiratory information can include use of a respiratory therapy system, a type of respiratory therapy device, a duration of respiratory therapy usage (e.g., how many hours per night a user uses their respiratory therapy system), a length of respiratory therapy usage (e.g., for what period of time, such as days, months, and/or years, a user has been using their respiratory therapy system), a sleep schedule, a duration of sleep, medical conditions, an apnea hypopnea index, a sleep score, etc.

The sources for the community-driven inputs can include content from one or more social media channels or platforms, one or more online shopping platforms, online news articles, product reviews (e.g., reviews by medical professionals and/or from other users), professional journals, videos, audio recordings, podcasts, one or more internet publications, one or more online feedback platforms that provide product feedback on one or more sleep-related and/or respiratory-related therapy devices and components, other types of online communities that related to one or more sleep-related and/or respiratory-related therapy devices and components, and various combinations thereof.

The one or more social medial channels or platforms can include messaging and/or video posting platforms (e.g., Facebook, Twitter, Tiktok, Youtube, Instagram, blogs, vlogs, etc.) chat or instant messaging platforms (e.g., Telegram, WhatsApp, WeChat, etc.), online discussion forums and webpages (e.g., Reddit, Quora, online article comment sections, etc.), and the like. The one or more online feedback platforms can include an application on a user device (e.g., a customer or patient engagement application such as myAir™), an internet platform (e.g., a website containing product reviews), a sleep/respiratory therapy device with online connectivity (e.g., a graphical user interface of a respiratory therapy device that sends information to a control system), and the like.

In some implementations, the control system can target search for content published by certain types of individuals or groups, such as one or more social media influencers, key opinion leaders, and other users (referred to herein as one or more second users) who have at least one commonality with the first user. Examples of key opinion leaders include medical professionals, clinician groups, and other predefined entities, such as trusted personalities or organizations whom consumers often rely on for guiding their purchasing decisions. For the one or more second users, the commonality between the first user and the one or more second users can be based on user information associated with the first user and the one or more second users, as discussed in more detail below.

According to some implementations, the data from the plurality community-driven inputs is identified by keyword-based searches on internet sources, such as using web crawling algorithm. For example, the control system can generate a crawl list of keyword terms (e.g., brand names, device models, frequently used words and phrases indicating quality and like/dislike, etc.). Furthermore, according to some implementations, the keyword-based search can be conducted on the internet at-large, on one or more predetermined internet sources (e.g., a group of websites dedicated to sleep health, a list of predetermined influencer social media accounts, or a list of medical journals), on a refined set of internet sources sorted by search criteria (e.g., a set of internet sources from a geographical region or in a specific language), or a combination of the foregoing search methods. Thus, data from the community-driven inputs can be identified via searching by keyword.

At step 1004, a measurement score is determined that ranks one or more sleep-related and/or respiratory-related devices/components based on the data being correlated to user information associated with the first user, each of the device model having a respective score rank. For example, according to some implementations, the sleep-related and/or respiratory-related equipment can include user interfaces, PAP devices, non-PAP devices, replacement parts and supplies, wearable devices, airable devices, nearable devices, and other equipment used in sleep and/or respiratory therapy. According to some implementations, the measurement score can be a direct measurement (e.g., a number of times that a product model has been purchased), an abstract index calculated based on a metric (e.g., a score between 0 and 100 indicating the quality of the product model), or a relative ranking between various product models (e.g., ranking a first type of user interface above a second type of user interface model to indicate one being superior over the other).

According to some implementations, the calculation of the measurement score can be based solely on the data from the sensors and/or the community-driven inputs to provide a generalized measurement score for all users. According to additional implementations, the calculation of the measurement score can be customized to the first user. For example, the measurement score can be based on a correlation between the data (i.e., data from the sensors and/or the community-driven inputs) and user information associated with the first user. It is noted that the user information is described herein with reference to the first user for whom the recommendation is being generated, but any description of the user information may also be applicable to any of the second users who are data sources for the community-driven inputs.

The user information associated with the first user can originate from a variety of different sources. In some implementations, the user information includes user-provided data (such as information inputted into a database by the first user, such as through a user device in communication with the database), an internet activity history associated with the first user, data generated by a user device associated with the first user (such as measurable data), or any combination thereof. The user information may also include data collected from the first user using the one or more sensors, data collected from online sources such as user profiles of social media accounts associated with the first user, or both. Thus, the user information can come from generally any source.

Specific types of user information can include a user device settings, user personal information, user sleep information, and user respiratory information (which can generally include any respiratory information associated with the first user that is generated by the one or more sensors as described above). The user personal information can include any suitable categories. For example, the personal information can include physical characteristics (e.g., facial characteristics, facial scan data, and skin texture), ethnic identity, geographical location, sex, age, gender identity, sexual orientation, interests, hobbies, likes, dislikes, preferences, lifestyle choices (e.g., smoking, alcohol user, dietary restrictions), user-provided data, an internet activity history associated with the first user, measured data that is generated by a user device associated with the first user, etc. The sleep information can include any suitable categories. For example, the sleep information can include a sleep diagnosis (e.g., of insomnia, restless leg syndrome, parasomnia, narcolepsy, circadian rhythm sleep-wake disorders, sleep apnea, etc.), a sleep schedule, a duration of sleep, restlessness, severity of sleep disorder, user device usage, use of a type of sleep enhancement device (e.g., sleep enhancement headband), etc.

Based on the data (from the sensors and/or the community-driven inputs) and the above-described user information, the measurement score can be calculated. According to some implementations, the measurement score can be based on factors such as the expected comfortableness and performance for the first user and the popularity of the devices or component among other users (e.g., the one or more second users) having commonalities with the first user. For example, the measurement score may reflect the expected comfortableness of a type of user interface for someone such as the first user who has high cheekbones (i.e., a specific facial characteristic) by comparing to one or more second users who also have high cheekbones. As another example, the measurement score can take into account the expected performance (e.g., effectiveness and reliability) of a model of PAP system for someone such as the first user who wears uses the respiratory therapy system nightly (i.e., a type of respiratory information) by comparing to one or more second users who also use their respiratory therapy system nightly. As a further example, the measurement score can take into account the popularity of a PAP system among one or more second users in the first user's geographical region who also use a PAP system, where the air tends to be cold and dry. According to some implementations, the popularity of a device/component can be measured by the frequency that the device/component appears in keyword searches. In some of these implementations, the keyword searches are limited to those originating from IP addresses from the first user's geographical region.

Thus, in some implementations, the measurement score is based on: (i) sensor data about the type of user interface gather from a user community; (ii) online information about the user interface generated by individuals and groups such as influencers, key opinion leaders, and one or more second users who have commonalities with the first user; and (iii) user information associated with the first user, which could be inputted by the first user or automatically generated based on data collected from the first user using sensors, one or more user devices associated with the first user, online sources such as user profiles of social media accounts associated with the first user, or any combination thereof.

In some implementations, the measurement score can include an overall score for the sleep-related and/or respiratory-related devices or component. Additionally, or alternatively, the measurement score can include one or more category scores, such as having a score for each category that the device/component is evaluated on (e.g., comfortableness, performance, and popularity). Other possible categories can include user interface size, user interface weight, power savings rating, etc. According to some implementations, the overall score can be based on the collection of category scores, such as by aggregation, average, weighted average, or any other scoring formula. Thus, in some implementations, in addition to the sensor data, the data from the community-driven inputs, the user information, or any combination thereof, the measurement score is further based on at least one of a comfortableness rating, a performance rating, a user interface size, a user interface weight, a power savings rating, and a frequency of mention in the plurality of community-driven inputs.

Furthermore, according to some implementations, the algorithm for calculating the measurement score can assign variable weights to factors when the measurement score is based on multiple factors (e.g., factors such as a collection of data from the sensors and the community-driven inputs and entries in the user information). For example, the measurement score for the type of user interface can be based on reviews gathered from multiple community-driven sources. For instance, an algorithm for calculating the measurement score can assign more weight to reviews of the user interface by a medical journal or a second user who has many commonalities with the first user. By contrast, the same algorithm can assign a lower weight to a review of the user interface posted by an influencer who has little history of reviewing user interfaces or another second user who has few commonalities with the first user.

At step 1006, suitable devices/components based on the measurement score are identified. For example, the control system identifies one or more suitable devices/components from the plurality of sleep-related and/or respiratory-related devices/components based on the respective score rank of each device/component. According to some implementations, the selection of the suitable devices/components is by ranking the plurality of devices/components by their respective measurement scores. According to some implementations, the selection can be based on ranking the overall measurement score of the devices/components. Alternatively, the selection can be based on ranking of one or more category scores, as previously described. For example, this can be accomplished by using a filtering function to isolate one or more desired category scores and ranking the category scores accordingly. According to some implementations, the suitable devices/components only includes a subset of the plurality of devices/components that pass one or more threshold criteria, such a minimum score (e.g., a minimum overall measurement score). Alternatively, the threshold criteria can include multiple score thresholds. For example, the list of suitable devices/components only includes devices/components that meet both a minimum comfortableness score and a minimum popularity score.

At step 1008, a recommendation is provided to the first user. For example, the control system provides or causes a recommendation to be provided to the first user. According to some implementations, the recommendation can be transmitted to a user device (e.g., a smartphone, a computer, or a wearable device) associated with the first user via a notification. The recommendation can recommend one or more of the suitable sleep-related and/or respiratory-related devices to the first user. According to some implementations, if the first user is already using a type of sleep-related and/or respiratory-related devices/components (such as a type of user interface), the recommendation notification can recommend one or more replacement models to the first user, which may be determined to be an improvement over the current model. In some implementations, the recommendation will include a link or button that allows the first user to purchase the recommended sleep-related and/or respiratory-related device/component.

According to some implementations, the control system can recognize a current model used by the first user based on the user information associated with the first user. This can include information inputted by the first user or information automatically gathered based on data collected from the first user using the one or more sensors, one or more user devices associated with the first user, or online sources such as user profiles of social media accounts associated with the first user. According to some implementations, the control system can also detect a decrease in satisfaction with a device/component by monitoring for keywords in the internet activity history of the first user and/or decreased usage of the current device/component based on the data from the plurality of sensors. In response to detecting the decrease in usage or the dissatisfaction, the control system can cause a notification to a user device associated with the first user, wherein the notification recommends replacement of the current device/component, such as by recommending the first user to replace a current user interface with the one or more suitable user interfaces, which may be a user interface that is improved relative to the current user interface.

In some implementations, method 1000 includes receiving a user rating from the first user on at least one of the plurality of community-driven inputs for the one or more suitable user interfaces. The method further includes adjusting the score rank of the one or more suitable user interfaces based on the user rating from the first user. This has the benefit of providing a mechanism for users who made purchases based on the recommendation to provide feedback and improve the recommendation.

In some implementations, the measurement score is further based on at least one of a comfortableness rating, a performance rating, an aesthetic rating, a claustrophobia rating, a profile rating, a price rating, a value rating, a noise rating, a community popularity rating (also referred to as a community sentiment rating, and which may be measured based on, for example, a frequency of mention and/or a frequency of positive feedback in the plurality of community-driven inputs), a successful switch rating, other types of ratings, and various combinations thereof. With respect to the successful switch rating, the level of success can be determined based on comparison of one or more quantifiable parameters from before and after the switch from a previous mask to a new mask. For instance, the comparison of such quantifiable parameters can include a comparison of the user rating of the previous mask to that of the new mask, or the changes in one or more sleep-related parameters after the user has switched from the previous mask to the new mask. The changes in sleep-related parameters can include, but is not limited to, increased therapy usage or reduced mask leakage as recorded by a respiratory therapy device or a user device. The rating may be determined or compared across masks of the same category (e.g., nasal, pillow, full face, cradle, ultra-compact full face, etc.)

In general, any of the data that is generated and/or used in conjunction with method 900 can also be used with method 1000. Similarly, any of the data that is generated and/or used in conjunction with method 1000 can also be used with method 900. For example, some of the data associated with the individual received at step 902 of method 900 may also be data that is received from the plurality of sensors at step 1002 of method 1000, and/or may include user information that is used at step 1004.

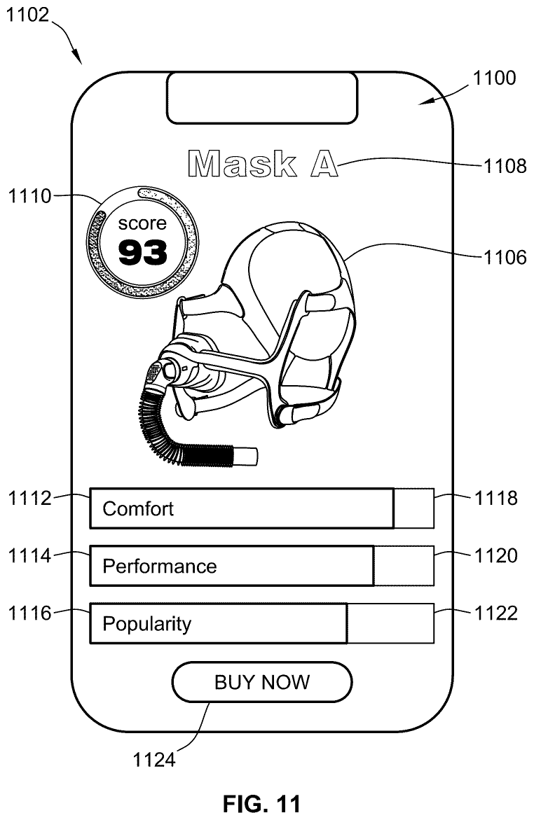

FIG. 11 depicts a user device 1102 presenting a recommendation regarding a user interface, in accordance with method 900 and/or method 1000. In some implementations, the user device 1102 can include a graphical user interface 1104 that shows the recommendation and/or provides information related to the recommendation. In some implementations, the graphical user interface 1104 displays a visual representation 1106 of the recommended device/component and/or a name or descriptor 1108 of the recommended device/component (e.g., User Interface A). In some implementations, the graphical user interface 1104 can show an overall measurement score 1110, which may be generated as part of method 1000. The overall measurement score 1110 can show the suitability of the recommended device/component. Additionally or alternatively, the graphical user interface 1104 can also show one or more indicators of various types of information that may have led to the recommendation and/or on which the recommended device/component was scored. For example, in the illustrated implementation, these indicators include a comfort indicator 1112, a performance indicator 1114, and a popularity indicator 1116, each accompanied by a corresponding rating bar 1118, 1120, 1122.

In some implementations, these indicators 1112, 1114, 1116 can indicate ratings/recommendations assigned to the recommended device/component by other individuals that are similar to the individual for whom the recommendation is being provided. In some implementations, the indicators 1112, 1114, 1116 indicate the individual measurement score of each category as determined according to method 1000. The various scores and ratings be represented numerically (e.g., having an overall measurement score of 93 as depicted in FIG. 11), graphically (e.g., having a rating bar that fills up to a percentage of the whole depending on the score), or by any other representation.

In some implementations, the graphical user interface 1104 can include a link (e.g., a hyperlink or an action button 1124) that, when activated, directs the user device to a purchase window for the recommended device/component. In some implementations, the purchase window can be opened in a browser or a separate application for online purchasing platforms such as ResMed, eBay, or Amazon.

In some implementations, method 900 and/or method 1000 (and/or any of the various implementations of method 900 and/or method 1000 described herein) can be implemented using a system for determining a user interface type (such as system 10). The system includes a control system (such as control system 200 of system 10) and a memory (such as memory device 204 of system 10). The control system includes one or more processors (such as processor 202 of control system 200). The memory has stored thereon machine-readable instructions. The control system is coupled to the memory, and method 900 and/or the method 1000 (and/or any of the various implementations of method 900 and/or method 1000 described herein) can be implemented when the machine-readable instructions in the memory are expected by at least one of the one or more processors of the control system.

Generally, method 900 and/or method 1000 can be implemented using a system (such as system 10) having a control system (such as control system 200 of system 10) with one or more processors (such as processor 202 of control system 200), and a memory (such as memory device 204 of system 10) storing machine-readable instructions. The control system can be coupled to the memory, and method 900 and/or method 1000 can be implemented when the machine-readable instructions are executed by at least one of the processors of the control system. Method 900 and/or method 1000 can also be implemented using a computer program product (such as a non-transitory computer readable medium) comprising instructions that when executed by a computer, cause the computer to carry out the steps of method 900 and/or method 1000.

### Alternative Implementations

Alternative Implementation 1. A method for generating recommendations related to use of a respiratory therapy system, the method comprising: receiving data associated with an individual who uses the respiratory therapy system; receiving data associated with a plurality of other individuals, each of the plurality of other individuals being users of a respective respiratory therapy system; and based at least in part on the received data, determining a recommended type of user interface to be worn by the individual during use of the respiratory therapy system by the individual.

Alternative Implementation 2. The method of Alternative Implementation 1, wherein the recommended type of user interface is selected from a plurality of types of user interface.

Alternative Implementation 3. The method of Alternative Implementation 2, wherein the recommended type of user interface is a type of user interface of the plurality of types of user interface that minimizes an amount of unintentional air leak when worn by the individual during use of the respiratory therapy system.

Alternative Implementation 4. The method of Alternative Implementation 2 or Alternative Implementation 3, wherein the recommended type of user interface is a type of user interface of the plurality of types of user interface that is predicted to be most comfortable for the individual.

Alternative Implementation 5. The method of Alternative Implementation 4, wherein a comfort of each of the plurality of types of user interfaces is based at least in part on a rating given to each of the plurality of types of user interfaces by one or more of the plurality of other individuals.

Alternative Implementation 6. The method of any one of Alternative Implementations 1 to 5, further comprising: determining a type of user interface worn by the individual during one or more prior uses of the respiratory therapy system; and in response to the type of user interface previously worn by the individual being different than the recommended type of user interface, sending a recommendation to the individual to switch to the recommended type of user interface during one or more future uses of the respiratory therapy system.

Alternative Implementation 7. The method of any one of Alternative Implementations 1 to 5, further comprising: determining a type of user interface worn by the individual during one or more prior uses of the respiratory therapy system; and in response to the type of user interface previously worn by the individual being identical to the recommended type of user interface, sending a recommendation to the individual to continue using the recommended type of user interface during one or more future uses of the respiratory therapy system.

Alternative Implementation 8. The method of Alternative Implementation 6 or Alternative Implementation 7, wherein determining the type of user interface worn by the individual during the one or more prior uses of the respiratory therapy system includes (i) receiving input from the individual indicative of the type of user interface worn by the individual during the one or more prior uses of the respiratory therapy system, (ii) analyzing data associated with the one or more prior uses of the respiratory therapy system to determine the type of user interface worn by the individual during the one or more prior uses of the respiratory therapy system, or (iii) both (i) and (ii).

Alternative Implementation 9. The method of any one of Alternative Implementations 1 to 8, wherein the data associated with each respective other individual of the plurality of other individuals includes (i) data associated with a user interface worn by the respective other individual and (ii) personal data associated with the respective other individual.

Alternative Implementation 10. The method of Alternative Implementation 9, wherein the personal data associated with the respective other individual is indicative of one or more physical characteristics of the respective other individual, an ambient temperature of a location of the respective other individual during use of the respective respiratory therapy system, an ambient humidity of the location of the respective other individual during use of the respective other respiratory therapy system, sleeping habits of the respective other individual, a severity of a respiratory condition of the respective other individual, a typical sleeping position of the respective other individual, an amount of movement of the respective other individual during one or more uses of the respective respiratory therapy system, a breathing path of the respective other individual during one or more uses of the respective respiratory therapy system, an ethnicity of the respective other individual, or any combination thereof.

Alternative Implementation 11. The method of Alternative Implementation 10, wherein the one or more physical characteristics of the respective other individual includes a size of a face of the respective other individual, a shape of the face of the respective other individual, a size of a mouth of the respective other individual, a shape of the mouth of the respective other individual, a size of a nose of the respective other individual, a shape of a nose of the respective other individual, a presence of facial hair on the respective other individual, one or more skin characteristics of the respective other individual, or any combination thereof.

Alternative Implementation 12. The method of Alternative Implementation 10 or Alternative Implementation 11, wherein the respiratory condition of the respective other individual is obstructive sleep apnea (OSA), and wherein the severity of the OSA is based on an apnea-hypopnea index (AHI) of the respective other individual.

Alternative Implementation 13. The method of any one of Alternative Implementations 10 to 12, wherein the typical sleeping position of the respective other individual is (i) the respective other individual lying on a back of the respective other individual or (ii) the respective other individual lying on a side of the respective other individual.

Alternative Implementation 14. The method of any one of Alternative Implementations 10 to 13, wherein the breathing path of the respective other individual is through a mouth of the respective other individual, through a nose of the respective other individual, or through both the mouth and the nose of the respective other individual.

Alternative Implementation 15. The method of any one of Alternative Implementations 9 to 14, wherein the data associated with the user interface worn by the respective other individual is indicative of a type of the user interface, a tightness of the user interface, a sleep position of the respective other individual during the use of the respective respiratory therapy system, a rating of the user interface given by the respective other individual, an amount of unintentional air leak out of the user interface worn by the respective other individual, or any combination thereof.

Alternative Implementation 16. The method of Alternative Implementation 15, wherein the tightness of the user interface is quantified using a numerical scale.

Alternative Implementation 17. The method of Alternative Implementation 16, wherein a first end of the numerical scale indicates that the user interface is under-tightened, and a second end of the numerical scale indicates that the user interface is over-tightened.

Alternative Implementation 18. The method of any one of Alternative Implementations 15 to 17, wherein the data associated with the user interface is indicative of one or more skin abnormalities of the individual following one or more sleep sessions, and wherein the tightness of the user interface is determined from the one or more skin abnormalities.

Alternative Implementation 19. The method of Alternative Implementation 18, wherein the one or more skin abnormalities includes one or more marks on a face of the individual caused by the user interface, a discoloration on the face of the individual caused by the user interface, or both.

Alternative Implementation 20. The method of any one of Alternative Implementations 1 to 19, further comprising identifying, based at least in part on the received data, one or more of the plurality of other individuals that match the individual, and wherein determining the recommended user interface type to be worn by the individual is based at least in part on the identification of the one or more of the plurality of other individuals that match the individual.

Alternative Implementation 21. The method of Alternative Implementation 20, wherein the matching one or more individuals share at least one of the one or more physical characteristics with the individual.

Alternative Implementation 22. The method of Alternative Implementation 20 or Alternative Implementation 21, wherein the matching one or more individuals have an identical severity of the respiratory condition as the individual, and identical typical sleeping position as the individual, an identical amount of movement during one or more uses of the respective respiratory therapy system as the individual, an identical breathing path during one or more uses of the respective respiratory therapy system as the individual, or any combination thereof.

Alternative Implementation 23. The method of any one of Alternative Implementations 1 to 22, further comprising: identifying, based at least in part on the received data, one or more of the plurality of other individuals that match the individual; and selecting the type of user interface worn by at least one of the matching other individuals as the recommended type of user interface to be worn by the individual during use of the respiratory therapy system.

Alternative Implementation 24. The method of any one of Alternative Implementations 20 to 23, wherein identifying the matching one or more other individuals includes identifying one or more of the plurality of other individuals who have (i) a similar nose as the individual, (ii) a similar mouth as the individual, (iii) a similar cheekbone height as the individual, (iv) a similar face width as the individual, (v) a similar amount of facial hair as the individual, or (v) any one of (i) through (iv).

Alternative Implementation 25. The method of Alternative Implementation 24, wherein the matching one or more other individuals with the similar nose as the individual have a similar nose size as the individual, a similar nose shape as the individual, a similar nose height as the individual, a similar nose width as the individual, a similar nose depth as the individual, or any combination thereof.

Alternative Implementation 26. The method of Alternative Implementation 24 or Alternative Implementation 25, wherein the matching one or more other individuals with the similar mouth as the individual have a similar mouth size as the individual, a similar mouth shape as the individual, a similar mouth height as the individual, a similar mouth width as the individual, or any combination thereof.

Alternative Implementation 27. The method of any one of Alternative Implementations 24 to 26, further comprising, in response to determining that the matching one or more other individuals use a first type of user interface, selecting the first type of user interface as the recommended type of user interface to be worn by the individual during the use of the respiratory therapy system.

Alternative Implementation 28. The method of any one of Alternative Implementations 24 to 26, further comprising, in response to determining that the matching one or more other individual do not use a first type of user interface, selecting a second type of user interface as the recommended type of user interface to be worn by the individual during the use of the respiratory therapy system.

Alternative Implementation 29. The method of Alternative Implementation 27 or Alternative Implementation 28, wherein the matching one or more other individuals have a similar nose as the individual.

Alternative Implementation 30. The method of any one of Alternative Implementations 20 to 29, wherein the matching one or more other individuals have an identical breathing path as the individual.

Alternative Implementation 31. The method of any one of Alternative Implementations 27 to 30, wherein the first type of user interface is a nasal pillow mask or a nasal mask, and the second type of user interface is a full-face mask.

Alternative Implementation 32. The method of Alternative Implementation 31, wherein the nasal pillow mask and the nasal mask do not cover a mouth of the individual, and wherein the full-face mask does cover the mouth of the individual.

Alternative Implementation 33. The method of any one of Alternative Implementations 23 to 32, further comprising, based at least in part on the data associated with the plurality of other individuals, providing a recommendation to the individual associated with a fit of the recommended type of user interface.

Alternative Implementation 34. The method of Alternative Implementation 33, wherein the recommendation associated with the fit of the recommended type of user interface includes a recommendation to adjust a tightness of the recommended type of user interface, a recommendation to use a specified level of tightness for the recommended type of user interface, a recommendation to adjust a size of the recommended type of user interface, a recommendation to use a specified size for the recommended type of user interface, a recommendation to adjust a position of the recommended type of user interface on a face of the individual, a recommendation to use a specified position of the recommended type of user interface on the face or the individual, or any combination thereof.

Alternative Implementation 35. The method of any one of Alternative Implementations 23 to 34, wherein identifying the matching one or more of the plurality of other individuals includes: determining an ambient environment of the individual during the use of the respiratory therapy system by the individual; and identifying one or more of the plurality of other individuals who have an ambient environment during the use of the respective respiratory therapy system that matches the ambient environment of the individual.

Alternative Implementation 36. The method of any one of Alternative Implementations 23 to 35, wherein the identifying the matching one or more of the plurality of other individuals includes: determining one or more physical characteristics of the individual; determining one or more physical characteristics of each of the plurality of other individuals; and identifying one or more of the plurality of other individuals who share at least one physical characteristic with the individual.

Alternative Implementation 37. The method of Alternative Implementation 36, wherein the one or more physical characteristics includes a size of a nose, a shape of a nose, a size of a mouth, a shape of a mouth, a height of cheekbones, a width of a face, a presence of facial hair, one or more skin characteristics, or any combination thereof.

Alternative Implementation 38. The method of any one of Alternative Implementations 1 to 37, wherein the data associated with the individual includes data associated with a face scan of the individual performed after one or more sleep sessions.

Alternative Implementation 39. The method of Alternative Implementation 38, further comprising: identifying one or more of the plurality of other individuals having a face scan that matches the face scan of the individual; and selecting the type of user interface worn by at least one of the other individuals having a matching face scan as the recommended type of user interface to be worn by the individual during user of the respiratory therapy system.

Alternative Implementation 40. The method of Alternative Implementation 38 or Alternative Implementation 39, wherein the data associated with the face scan of the individual includes data reproducible as one or more images of a face of the individual, data reproducible as one or more videos of the face of the individual, data associated with one or more anatomical characteristics of the face of the individual, data associated with one or more depth measurements of the face of the individual, or any combination thereof.

Alternative Implementation 41. The method of Alternative Implementation 40, wherein the data associated with the one or more anatomical characteristics of the face of the individual includes data associated with a height of a nose of the individual, a width of the nose of the individual, a depth of the nose of the individual, a height of a mouth of the individual, a width of the mouth of the individual, a distance between the nose of the individual and a mouth of the individual, or any combination thereof.

Alternative Implementation 42. The method of Alternative Implementation 40 or Alternative Implementation 41, wherein at least a portion of the data associated with the face scan of the individual is generated with a smartphone of the individual.

Alternative Implementation 43. The method of any one of Alternative Implementations 38 to 42, wherein the data associated with each respective other individual of the plurality of other individuals includes data associated with a face scan of the respective other individual.

Alternative Implementation 44. The method of Alternative Implementation 43, further comprising identifying, based at least in part on the data associated with the face scan of the individual and the data associated with the face scan of the plurality of other individuals, one or more of the plurality of other individuals that match the individual.

Alternative Implementation 45. The method of Alternative Implementation 44, further comprising selecting the type of user interface worn by at least one of the matching other individuals as the recommended type of user interface to be worn by the individual during use of the respiratory therapy system.

Alternative Implementation 46. The method of Alternative Implementation 45, further comprising estimating an unintentional leak of the recommended type of user interface when worn by the individual during use of the respiratory therapy system, based at least in part on the face scan of the individual.

Alternative Implementation 47. The method of Alternative Implementation 46, wherein estimating the unintentional leak is further based at least in part on the face scan of the plurality of other individuals, a scan of the recommended type of user interface, or both.

Alternative Implementation 48. The method of any one of Alternative Implementations 1 to 47, further comprising: determining a value of one or more sleep metrics of the individual;
determining a value of the one or more sleep metrics for at least one of the plurality of other individuals; and taking an action based at least in part on the determined values.

Alternative Implementation 49. The method of Alternative Implementation 48, wherein the action includes transmitting to the individual a recommendation to use a specific type of user interface, a recommendation to change the type of user interface being used, a recommendation to adjust a fit of the user interface being worn, a recommendation to adjust one or more settings on the respiratory therapy system of the individual, a recommendation to use a personalized therapy algorithm with the respiratory therapy system of the individual, or any combination thereof.

Alternative Implementation 50. The method of any one of Alternative Implementations 1 to 49, further comprising: inputting the received data into a trained machine learning algorithm; and receiving from the trained machine learning algorithm the recommended type of user interface to be worn by the individual during use of the respiratory therapy system.

Alternative Implementation 51. A method for generating recommendations related to use of respiratory therapy system, the method comprising: receiving data from at least one of a plurality of sensors and at least one of a plurality of community-driven inputs, wherein the data from the plurality of sensors is objectively determined and the data from the plurality of community-driven inputs is subjectively determined; determining a measurement score that ranks a plurality of user interfaces based on the data being correlated to user information associated with a first user, each of the user interfaces having a respective score rank; identifying one or more suitable user interfaces from the plurality of user interfaces based on the respective score rank of each of the user interfaces; and providing a recommendation for the first user to use at least one of the one or more suitable user interfaces.

Alternative Implementation 52. The method of Alternative Implementation 51, wherein the plurality of community-driven inputs includes content on a social media channel, content on an online feedback platform, or both, the content generated by a social media influencer, a key opinion leader, a second user having at least one commonality with the first user based on the user information, or any combination thereof.

Alternative Implementation 53. The method of Alternative Implementation 52, wherein the user information includes a user facial characteristic, a user ethnic identity, a user age, a user gender, a user geographical location.

Alternative Implementation 54. The method of Alternative Implementation 52 or Alternative Implementation 53, wherein the key opinion leader includes at least one of a medical professional, a clinician group, and a predefined entity.

Alternative Implementation 55. The method of any one of Alternative Implementations 52 to 54, wherein the user information associated with the first user includes user-provided data, an internet activity history associated with the first user, measured data generated by a user device associated with the first user, or any combination thereof.

Alternative Implementation 56. The method of any one of Alternative Implementations 51 to 55, wherein the providing the recommendation includes providing a recommendation for the first user to replace a current user interface with the one or more suitable user interfaces.

Alternative Implementation 57. The method of any one of Alternative Implementations 51 to 56, further comprising searching by keyword to identify the data from the plurality of community-driven inputs.

Alternative Implementation 58. The method of Alternative Implementation 57, wherein the data from the plurality of community-driven inputs includes a frequency that a user interface of the plurality user interfaces appears in keyword search.

Alternative Implementation 59. The method of any one of Alternative Implementations 51 to 58, wherein the data from the plurality of sensors include at least one of facial scan data, audio data, user interface usage data, user interface leakage data, and user interface noise data.

Alternative Implementation 60. The method of any one of Alternative Implementations 51 to 59, wherein the plurality of sensors include at least one of a pressure sensor, a flow rate sensor, a temperature sensor, a motion sensor, a microphone, a speaker, a radio-frequency (RF) receiver, a RF transmitter, a camera, an infrared sensor, a photo-plethysmogram (PPG) sensor, an electrocardiogram (ECG) sensor, an electroencephalography (EEG) sensor, a capacitive sensor, a force sensor, a strain gauge sensor, an electromyography (EMG) sensor, an oxygen sensor, an analyte sensor, a moisture sensor, a LiDAR sensor, a facial scanner, a blood oxygen level sensor, a blood pressure sensor, a heart rate sensor, a photodetector, an air pressure sensor, and a device usage tracker.

Alternative Implementation 61. The method of any one of Alternative Implementations 51 to 60, wherein the providing a recommendation includes: detecting, by a control system, a decrease in usage of the current user interface in the data from the plurality of sensors or a keyword in an internet activity history of the first user indicative of dissatisfaction with the current user interface; and causing transmission, by a control system, a notification to a user device associated with the first user, wherein the notification recommends replacement of the current user interface with the one or more suitable user interfaces.

Alternative Implementation 62. The method of any one of Alternative Implementations 51 to 61, further comprising: receiving a user rating from the first user on at least one of the plurality of community-driven inputs for the one or more suitable user interfaces; and adjusting the score rank of the one or more suitable user interfaces based on the user rating from the first user.

Alternative Implementation 63. The method of any one of Alternative Implementations 51 to 62, wherein the measurement score that ranks the plurality of user interfaces is further based on at least one of a comfortableness rating, a performance rating, an aesthetic rating, a claustrophobia rating, a price rating, a value rating, a noise rating, a community popularity rating, a user interface size, a user interface weight, and a power savings rating.

Alternative Implementation 64. A system for generating recommendations related to use of a respiratory therapy system, the system comprising: a control system including one or more processors; and a memory having stored thereon machine-readable instructions; wherein the control system is coupled to the memory, and the method of any one of Alternative Implementations 1 to 63 is implemented when the machine-readable instructions in the memory are executed by at least one of the one or more processors of the control system.

Alternative Implementation 65. A system for generating recommendations related to use of a respiratory therapy system, the system including a control system having one or more processors configured to implement the method of any one of Alternative Implementations 1 to 63.

Alternative Implementation 66. A computer program product comprising instructions which, when executed by a computer, cause the computer to carry out the method of any one of Alternative Implementations 1 to 63.

Alternative Implementation 67. The computer program product of Alternative Implementation 66, wherein the computer program product is a non-transitory computer readable medium.

Alternative Implementation 68. A system for generating recommendations related to use of a respiratory therapy system, the system comprising: an electronic interface configured to receive data associated with a sleep session of an individual; a memory storing machine-readable instructions; and a control system including one or more processors configured to execute the machine-readable instructions to: receive the data associated with the individual who uses the respiratory therapy system; receive data associated with a plurality of other individuals, each of the plurality of other individuals being users of a respective respiratory therapy system; and based at least in part on the received data, determine a recommended type of user interface to be worn by the individual during use of the respiratory therapy system by the individual.

Alternative Implementation 69. The system of Alternative Implementation 68, wherein the recommended type of user interface is selected from a plurality of types of user interface.

Alternative Implementation 70. The system of Alternative Implementation 69, wherein the recommended type of user interface is a type of user interface of the plurality of types of user interface that minimizes an amount of unintentional air leak when worn by the individual during use of the respiratory therapy system.

Alternative Implementation 71. The system of Alternative Implementation 69 or Alternative Implementation 70, wherein the recommended type of user interface is a type of user interface of the plurality of types of user interface that is predicted to be most comfortable for the individual.

Alternative Implementation 72. The system of Alternative Implementation 71, wherein a comfort of each of the plurality of types of user interfaces is based at least in part on a rating given to each of the plurality of types of user interfaces by one or more of the plurality of other individuals.

Alternative Implementation 73. The system of any one of Alternative Implementations 68 to 72, wherein the one or more processors are further configured to execute the machine-readable instructions to: determine a type of user interface worn by the individual during one or more prior uses of the respiratory therapy system; and in response to the type of user interface previously worn by the individual being different than the recommended type of user interface, send a recommendation to the individual to switch to the recommended type of user interface during one or more future uses of the respiratory therapy system.

Alternative Implementation 74. The system of any one of Alternative Implementations 68 to 73, wherein the one or more processors are further configured to execute the machine-readable instructions to: determine a type of user interface worn by the individual during one or more prior uses of the respiratory therapy system; and in response to the type of user interface previously worn by the individual being identical to the recommended type of user interface, send a recommendation to the individual to continue using the recommended type of user interface during one or more future uses of the respiratory therapy system.

Alternative Implementation 75. The system of Alternative Implementation 73 or Alternative Implementation 74, wherein determining the type of user interface worn by the individual during the one or more prior uses of the respiratory therapy system includes (i) receiving input from the individual indicative of the type of user interface worn by the individual during the one or more prior uses of the respiratory therapy system, (ii) analyzing data associated with the one or more prior uses of the respiratory therapy system to determine the type of user interface worn by the individual during the one or more prior uses of the respiratory therapy system, or (iii) both (i) and (ii).

Alternative Implementation 76. The system of any one of Alternative Implementations 68 to 75, wherein the data associated with each respective other individual of the plurality of other individuals includes (i) data associated with a user interface worn by the respective other individual and (ii) personal data associated with the respective other individual.

Alternative Implementation 77. The system of Alternative Implementation 76, wherein the personal data associated with the respective other individual is indicative of one or more physical characteristics of the respective other individual, an ambient temperature of a location of the respective other individual during use of the respective respiratory therapy system, an ambient humidity of the location of the respective other individual during use of the respective other respiratory therapy system, sleeping habits of the respective other individual, a severity of a respiratory condition of the respective other individual, a typical sleeping position of the respective other individual, an amount of movement of the respective other individual during one or more uses of the respective respiratory therapy system, a breathing path of the respective other individual during one or more uses of the respective respiratory therapy system, an ethnicity of the respective other individual, or any combination thereof.

Alternative Implementation 78. The system of Alternative Implementation 77, wherein the one or more physical characteristics of the respective other individual includes a size of a face of the respective other individual, a shape of the face of the respective other individual, a size of a mouth of the respective other individual, a shape of the mouth of the respective other individual, a size of a nose of the respective other individual, a shape of a nose of the respective other individual, a presence of facial hair on the respective other individual, one or more skin characteristics of the respective other individual, or any combination thereof.

Alternative Implementation 79. The system of Alternative Implementation 77 or Alternative Implementation 78, wherein the respiratory condition of the respective other individual is obstructive sleep apnea (OSA), and wherein the severity of the OSA is based on an apnea-hypopnea index (AHI) of the respective other individual.

Alternative Implementation 80. The system of any one of Alternative Implementations 77 to 79, wherein the typical sleeping position of the respective other individual is (i) the respective other individual lying on a back of the respective other individual or (ii) the respective other individual lying on a side of the respective other individual.

Alternative Implementation 81. The system of any one of Alternative Implementations 77 to 80, wherein the breathing path of the respective other individual is through a mouth of the respective other individual, through a nose of the respective other individual, or through both the mouth and the nose of the respective other individual.

Alternative Implementation 82. The system of any one of Alternative Implementations 76 to 81, wherein the data associated with the user interface worn by the respective other individual is indicative of a type of the user interface, a tightness of the user interface, a sleep position of the respective other individual during the use of the respective respiratory therapy system, a rating of the user interface given by the respective other individual, an amount of unintentional air leak out of the user interface worn by the respective other individual, or any combination thereof.

Alternative Implementation 83. The system of Alternative Implementation 82, wherein the tightness of the user interface is quantified using a numerical scale.

Alternative Implementation 84. The system of Alternative Implementation 83, wherein a first end of the numerical scale indicates that the user interface is under-tightened, and a second end of the numerical scale indicates that the user interface is over-tightened.

Alternative Implementation 85. The system of any one of Alternative Implementations 82 to 84, wherein the data associated with the user interface is indicative of one or more skin abnormalities of the individual following one or more sleep sessions, and wherein the tightness of the user interface is determined from the one or more skin abnormalities.

Alternative Implementation 86. The system of Alternative Implementation 85, wherein the one or more skin abnormalities includes one or more marks on a face of the individual caused by the user interface, a discoloration on the face of the individual caused by the user interface, or both.

Alternative Implementation 87. The system of any one of Alternative Implementations 68 to 86, wherein the one or more processors are further configured to execute the machine-readable instructions to identify, based at least in part on the received data, one or more of the plurality of other individuals that match the individual, and wherein determining the recommended user interface type to be worn by the individual is based at least in part on the identification of the one or more of the plurality of other individuals that match the individual.

Alternative Implementation 88. The system of Alternative Implementation 87, wherein the matching one or more individuals share at least one of the one or more physical characteristics with the individual.

Alternative Implementation 89. The system of Alternative Implementation 87 or Alternative Implementation 88, wherein the matching one or more individuals have an identical severity of the respiratory condition as the individual, and identical typical sleeping position as the individual, an identical amount of movement during one or more uses of the respective respiratory therapy system as the individual, an identical breathing path during one or more uses of the respective respiratory therapy system as the individual, or any combination thereof.

Alternative Implementation 90. The system of any one of Alternative Implementations 68 to 89, wherein the one or more processors are further configured to execute the machine-readable instructions to: identify, based at least in part on the received data, one or more of the plurality of other individuals that match the individual; and select the type of user interface worn by at least one of the matching other individuals as the recommended type of user interface to be worn by the individual during use of the respiratory therapy system.

Alternative Implementation 91. The system of any one of Alternative Implementations 87 to 90, wherein identifying the matching one or more other individuals includes identifying one or more of the plurality of other individuals who have (i) a similar nose as the individual, (ii) a similar mouth as the individual, (iii) a similar cheekbone height as the individual, (iv) a similar face width as the individual, (v) a similar amount of facial hair as the individual, or (v) any one of (i) through (iv).

Alternative Implementation 92. The system of Alternative Implementation 91, wherein the matching one or more other individuals with the similar nose as the individual have a similar nose size as the individual, a similar nose shape as the individual, a similar nose height as the individual, a similar nose width as the individual, a similar nose depth as the individual, or any combination thereof.

Alternative Implementation 93. The system of Alternative Implementation 91 or Alternative Implementation 92, wherein the matching one or more other individuals with the similar mouth as the individual have a similar mouth size as the individual, a similar mouth shape as the individual, a similar mouth height as the individual, a similar mouth width as the individual, or any combination thereof.

Alternative Implementation 94. The system of any one of Alternative Implementations 91 to 93, wherein the one or more processors are further configured to execute the machine-readable instructions to, in response to determining that the matching one or more other individuals use a first type of user interface, select the first type of user interface as the recommended type of user interface to be worn by the individual during the use of the respiratory therapy system.

Alternative Implementation 95. The system of any one of Alternative Implementations 91 to 94, wherein the one or more processors are further configured to execute the machine-readable instructions to, in response to determining that the matching one or more other individual do not use a first type of user interface, select a second type of user interface as the recommended type of user interface to be worn by the individual during the use of the respiratory therapy system.

Alternative Implementation 96. The system of Alternative Implementation 94 or Alternative Implementation 95, wherein the matching one or more other individuals have a similar nose as the individual.

Alternative Implementation 97. The system of any one of Alternative Implementations 87 to 96, wherein the matching one or more other individuals have an identical breathing path as the individual.

Alternative Implementation 98. The system of any one of Alternative Implementations 94 to 97, wherein the first type of user interface is a nasal pillow mask or a nasal mask, and the second type of user interface is a full-face mask.

Alternative Implementation 99. The system of Alternative Implementation 98, wherein the nasal pillow mask and the nasal mask do not cover a mouth of the individual, and wherein the full-face mask does cover the mouth of the individual.

Alternative Implementation 100. The system of any one of Alternative Implementations 90 to 99, wherein the one or more processors are further configured to execute the machine-readable instructions to, based at least in part on the data associated with the plurality of other individuals, provide a recommendation to the individual associated with a fit of the recommended type of user interface.

Alternative Implementation 101. The system of Alternative Implementation 100, wherein the recommendation associated with the fit of the recommended type of user interface includes a recommendation to adjust a tightness of the recommended type of user interface, a recommendation to use a specified level of tightness for the recommended type of user interface, a recommendation to adjust a size of the recommended type of user interface, a recommendation to use a specified size for the recommended type of user interface, a recommendation to adjust a position of the recommended type of user interface on a face of the individual, a recommendation to use a specified position of the recommended type of user interface on the face or the individual, or any combination thereof.

Alternative Implementation 102. The system of any one of Alternative Implementations 90 to 101, wherein identifying the matching one or more of the plurality of other individuals includes: determining an ambient environment of the individual during the use of the respiratory therapy system by the individual; and identifying one or more of the plurality of other individuals who have an ambient environment during the use of the respective respiratory therapy system that matches the ambient environment of the individual.

Alternative Implementation 103. The system of any one of Alternative Implementations 90 to 102, wherein the identifying the matching one or more of the plurality of other individuals includes: determining one or more physical characteristics of the individual; determining one or more physical characteristics of each of the plurality of other individuals; and identifying one or more of the plurality of other individuals who share at least one physical characteristic with the individual.

Alternative Implementation 104. The system of Alternative Implementation 103, wherein the one or more physical characteristics includes a size of a nose, a shape of a nose, a size of a mouth, a shape of a mouth, a height of cheekbones, a width of a face, a presence of facial hair, one or more skin characteristics, or any combination thereof.

Alternative Implementation 105. The system of any one of Alternative Implementations 68 to 104, wherein the data associated with the individual includes data associated with a face scan of the individual performed after one or more sleep sessions.

Alternative Implementation 106. The system of Alternative Implementation 105, wherein the one or more processors are further configured to execute the machine-readable instructions to: identify one or more of the plurality of other individuals having a face scan that matches the face scan of the individual; and select the type of user interface worn by at least one of the other individuals having a matching face scan as the recommended type of user interface to be worn by the individual during user of the respiratory therapy system.

Alternative Implementation 107. The system of Alternative Implementation 105 or Alternative Implementation 106, wherein the data associated with the face scan of the individual includes data reproducible as one or more images of a face of the individual, data reproducible as one or more videos of the face of the individual, data associated with one or more anatomical characteristics of the face of the individual, data associated with one or more depth measurements of the face of the individual, or any combination thereof.

Alternative Implementation 108. The system of Alternative Implementation 107, wherein the data associated with the one or more anatomical characteristics of the face of the individual includes data associated with a height of a nose of the individual, a width of the nose of the individual, a depth of the nose of the individual, a height of a mouth of the individual, a width of the mouth of the individual, a distance between the nose of the individual and a mouth of the individual, or any combination thereof.

Alternative Implementation 109. The system of Alternative Implementation 107 or Alternative Implementation 108, wherein at least a portion of the data associated with the face scan of the individual is generated with a smartphone of the individual.

Alternative Implementation 110. The system of any one of Alternative Implementations 105 to 109, wherein the data associated with each respective other individual of the plurality of other individuals includes data associated with a face scan of the respective other individual.

Alternative Implementation 111. The system of Alternative Implementation 110, wherein the one or more processors are further configured to execute the machine-readable instructions to: identify, based at least in part on the data associated with the face scan of the individual and the data associated with the face scan of the plurality of other individuals, one or more of the plurality of other individuals that match the individual; and select the type of user interface worn by at least one of the matching other individuals as the recommended type of user interface to be worn by the individual during use of the respiratory therapy system.

Alternative Implementation 112. The system of Alternative Implementation 111, further comprising estimating an unintentional leak of the recommended type of user interface when worn by the individual during use of the respiratory therapy system, based at least in part on the face scan of the individual.

Alternative Implementation 113. The system of Alternative Implementation 112, wherein estimating the unintentional leak is further based at least in part on the face scan of the plurality of other individuals, a scan of the recommended type of user interface, or both.

Alternative Implementation 114. The system of any one of Alternative Implementations 68 to 113, wherein the one or more processors are further configured to execute the machine-readable instructions to: determine a value of one or more sleep metrics of the individual; determine a value of the one or more sleep metrics for at least one of the plurality of other individuals; and take an action based at least in part on the determined values.

Alternative Implementation 115. The system of Alternative Implementation 114, wherein the action includes transmitting to the individual a recommendation to use a specific type of user interface, a recommendation to change the type of user interface being used, a recommendation to adjust a fit of the user interface being worn, a recommendation to adjust one or more settings on the respiratory therapy system of the individual, a recommendation to use a personalized therapy algorithm with the respiratory therapy system of the individual, or any combination thereof.

Alternative Implementation 116. The system of any one of Alternative Implementations 68 to 115, wherein the one or more processors are further configured to execute the machine-readable instructions to: input the received data into a trained machine learning algorithm; and receive from the trained machine learning algorithm the recommended type of user interface to be worn by the individual during use of the respiratory therapy system.

Alternative Implementation 117. A system for generating recommendations related to use of a respiratory therapy system, the system comprising: a database, wherein the database is configured to store user information associated with a first user of the respiratory therapy system; and a control system communicatively coupled to the database, wherein the control system is configured to: receive, by the control system, data from at least one of a plurality of sensors and a plurality of social media channels, wherein the data from the plurality of sensors is objectively determined and the data from the plurality of social media channels is subjectively determined by one or more of a social media influencer and a key opinion leader; determine, by the control system, a measurement score that ranks a plurality of user interfaces based on the data being correlated to the user information associated with the first user, each of the user interfaces having a respective score rank; identify, by the control system, one or more suitable user interfaces from the plurality of user interfaces based on the respective score rank of each of the user interfaces; and provide, by the control system, a recommendation for the first user to use at least one of the one or more suitable user interfaces.

Alternative Implementation 118. The system of Alternative Implementation 117, wherein the plurality of community-driven inputs include one or more of a shared content on a social media channel or a product review platform by one or more of a social media influencer, a key opinion leader, and a second user having at least one commonality with the first user based on the user information.

Alternative Implementation 119. The system of Alternative Implementation 117 or Alternative Implementation 118, wherein the user information includes a user facial characteristic, a user ethnic identity, a user age, a user gender, a user geographical location.

Alternative Implementation 120. The system of any one of Alternative Implementations 117 to 119, wherein the key opinion leader includes at least one of a medical professional, a second user, a clinician group, and a predefined entity.

Alternative Implementation 121. The system of any one of Alternative Implementations 117 to 120, wherein the control system is further configured to search, by the control system, keyword to identify the data from the plurality of community-driven inputs.

Alternative Implementation 122. The system of Alternative Implementation 121, wherein the data from the plurality of community-driven inputs includes a frequency that a user interface of the plurality user interfaces appears in keyword search.

Alternative Implementation 123. The system of any one of Alternative Implementations 117 to 122, wherein the user information associated with the first user includes at least one of user-provided data, an internet activity history associated with the first user, and measured data generated by a user device associated with the first user.

Alternative Implementation 124. The system of any one of claims 117 to 123, wherein the providing the recommendation includes providing a recommendation for the first user to replace a current user interface with the one or more suitable user interfaces.

Alternative Implementation. The system of any one of claims 117 to 124, wherein the data from the plurality of sensors include at least one of facial scan data, audio data, user interface usage data, user interface leakage data, and user interface noise data.

Alternative Implementation 126. The system of any one of claims 117 to 125, wherein the plurality of sensors include at least one of a pressure sensor, a flow rate sensor, a temperature sensor, a motion sensor, a microphone, a speaker, a radio-frequency (RF) receiver, a RF transmitter, a camera, an infrared sensor, a photo-plethysmogram (PPG) sensor, an electrocardiogram (ECG) sensor, an electroencephalography (EEG) sensor, a capacitive sensor, a force sensor, a strain gauge sensor, an electromyography (EMG) sensor, an oxygen sensor, an analyte sensor, a moisture sensor, a LiDAR sensor, a facial scanner, a blood oxygen level sensor, a blood pressure sensor, a heart rate sensor, a photodetector, an air pressure sensor, and a device usage tracker.

Alternative Implementation 127. The system of any one of claims 117 to 126, wherein the control system is further configured, to provide the recommendation, to: detect a decrease in usage of the current user interface in the data from the plurality of sensors or a keyword in an internet activity history of the first user indicative of dissatisfaction with the current user interface; and cause transmission of a notification to a user device associated with the first user, wherein the notification recommends replacement of the current user interface with the one or more suitable user interfaces.

Alternative Implementation 128. The system of any one of claims 117 to 127, wherein the control system is further configured to: receive a user rating from the first user on at least one of the plurality of community-driven inputs for the one or more suitable user interfaces; and adjust the score rank of the one or more suitable user interfaces based on the user rating from the first user.

Alternative Implementation 129. The system of any one of claims 117 to 128, wherein the measurement score that ranks the plurality of user interfaces is further based on at least one of a comfortableness rating, a performance rating, an aesthetic rating, a claustrophobia rating, a price rating, a value rating, a noise rating, a community popularity rating, a user interface size, a user interface weight, and a power savings rating.

One or more elements or aspects or steps, or any portion(s) thereof, from one or more of any of the Alternative Implementations and/or claims herein can be combined with one or more elements or aspects or steps, or any portion(s) thereof, from one or more of any of the other Alternative Implementations and/or claims herein or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method for generating recommendations related to use of a respiratory therapy system, the method comprising:
receiving data associated with an individual who uses the respiratory therapy system;
receiving data associated with a plurality of other individuals, each of the plurality of other individuals being users of a respective respiratory therapy system; and
based at least in part on the received data, determining a recommended type of user interface to be worn by the individual during use of the respiratory therapy system by the individual.

2. (canceled)

3. The method of claim 1, wherein the recommended type of user interface is a type of user interface of a plurality of types of user interface that (i) minimizes an amount of unintentional air leak when worn by the individual during use of the respiratory therapy system, (ii) is predicted to be most comfortable for the individual, or (iii) both (i) and (ii).

4-5. (canceled)

6. The method of claim 1, further comprising:
determining a type of user interface worn by the individual during one or more prior uses of the respiratory therapy system; and
(i) in response to the type of user interface previously worn by the individual being different than the recommended type of user interface, sending a recommendation to the individual to switch to the recommended type of user interface during one or more future uses of the respiratory therapy system;
(ii) in response to the type of user interface previously worn by the individual being identical to the recommended type of user interface, sending a recommendation to the individual to continue using the recommended type of user interface during one or more future uses of the respiratory therapy system; or
(iii) both (i) and (ii).

7. (canceled)

8. The method of claim 6, wherein determining the type of user interface worn by the individual during the one or more prior uses of the respiratory therapy system includes (i) receiving input from the individual indicative of the type of user interface worn by the individual during the one or more prior uses of the respiratory therapy system, (ii) analyzing data associated with the one or more prior uses of the respiratory therapy system to determine the type of user interface worn by the individual during the one or more prior uses of the respiratory therapy system, or (iii) both (i) and (ii).

9. The method of claim 1, wherein the data associated with each respective other individual of the plurality of other individuals includes (i) data associated with a user interface worn by the respective other individual and (ii) personal data associated with the respective other individual.

10-19. (canceled)

20. The method of claim 1, further comprising identifying, based at least in part on the received data, one or more of the plurality of other individuals that match the individual, and wherein determining the recommended user interface type to be worn by the individual is based at least in part on the identification of the one or more of the plurality of other individuals that match the individual.

21. The method of claim 20, wherein the matching one or more individuals (i) share at least one of the one or more physical characteristics with the individual (ii) have an identical severity of the respiratory condition as the individual, (iii) have an identical typical sleeping position as the individual, (iv) have an identical amount of movement during one or more uses of the respective respiratory therapy system as the individual, (v) have an identical breathing path during one or more uses of the respective respiratory therapy system as the individual, or (vi) any combination of (i)-(v).

22-26. (canceled)

27. The method of claim 20, further comprising:
in response to determining that the matching one or more other individuals use a first type of user interface, selecting the first type of user interface as the recommended type of user interface to be worn by the individual during the use of the respiratory therapy system; or
in response to determining that the matching one or more other individual do not use a first type of user interface, selecting a second type of user interface as the recommended type of user interface to be worn by the individual during the use of the respiratory therapy system.

28-34. (canceled)

35. The method of claim 20, wherein identifying the matching one or more of the plurality of other individuals includes:
determining (i) an ambient environment of the individual during the use of the respiratory therapy system by the individual, (ii) one or more physical characteristics of the individual, or (iii) both (i) and (ii); and
identifying one or more of the plurality of other individuals who (i) have an ambient environment during the use of the respective respiratory therapy system that matches the ambient environment of the individual, (ii) share at least one physical characteristic with the individual, or (iii) both (i) and (ii).

36-38. (canceled)

39. The method of claim 1, wherein the data associated with the individual includes data associated with a face scan of the individual performed after one or more sleep sessions and wherein the data associated with each respective other individual includes data associated with a face scan of the respective other individual, and wherein the method further comprises:
identifying one or more of the plurality of other individuals having a face scan that matches the face scan of the individual; and
selecting the type of user interface worn by at least one of the other individuals having a matching face scan as the recommended type of user interface to be worn by the individual during user of the respiratory therapy system.

40-45. (canceled)

46. The method of claim 39, further comprising estimating an unintentional leak of the recommended type of user interface when worn by the individual during use of the respiratory therapy system, based at least in part on the face scan of the individual, the face scan of the plurality of other individuals, a scan of the recommended type of user interface, or any combination thereof.

47. (canceled)

48. The method of claim 1, further comprising:
determining a value of one or more sleep metrics of the individual;
determining a value of the one or more sleep metrics for at least one of the plurality of other individuals; and
taking an action based at least in part on the determined values, the action including transmitting to the individual a recommendation to use a specific type of user interface, a recommendation to change the type of user interface being used, a recommendation to adjust a fit of the user interface being worn, a recommendation to adjust one or more settings on the respiratory therapy system of the individual, a recommendation to use a personalized therapy algorithm with the respiratory therapy system of the individual, or any combination thereof.

49. (canceled)

50. The method of claim 1, further comprising:
inputting the received data into a trained machine learning algorithm; and
receiving from the trained machine learning algorithm the recommended type of user interface to be worn by the individual during use of the respiratory therapy system.

51. A method for generating recommendations related to use of respiratory therapy system, the method comprising:
receiving data from at least one of a plurality of sensors and at least one of a plurality of community-driven inputs, wherein the data from the plurality of sensors is objectively determined and the data from the plurality of community-driven inputs is subjectively determined;
determining a measurement score that ranks a plurality of user interfaces based on the data being correlated to user information associated with a first user, each of the user interfaces having a respective score rank;
identifying one or more suitable user interfaces from the plurality of user interfaces based on the respective score rank of each of the user interfaces; and
providing a recommendation for the first user to use at least one of the one or more suitable user interfaces.

52. The method of claim 51, wherein the plurality of community-driven inputs includes content on a social media channel, content on an online feedback platform, or both, the content generated by a social media influencer, a key opinion leader, a second user having at least one commonality with the first user based on the user information, or any combination thereof.

53-56. (canceled)

57. The method of claim 51, further comprising searching by keyword to identify the data from the plurality of community-driven inputs.

58. The method of claim 57, wherein the data from the plurality of community-driven inputs includes a frequency that a user interface of the plurality user interfaces appears in keyword search.

59-60. (canceled)

61. The method of claim 51, wherein the providing a recommendation includes:
detecting, by a control system, a decrease in usage of the current user interface in the data from the plurality of sensors or a keyword in an internet activity history of the first user indicative of dissatisfaction with the current user interface; and
causing transmission, by a control system, a notification to a user device associated with the first user, wherein the notification recommends replacement of the current user interface with the one or more suitable user interfaces.

62. The method of claim 51, further comprising:
receiving a user rating from the first user on at least one of the plurality of community-driven inputs for the one or more suitable user interfaces; and
adjusting the score rank of the one or more suitable user interfaces based on the user rating from the first user.

63. The method of claim 51, wherein the measurement score that ranks the plurality of user interfaces is further based on at least one of a comfortableness rating, a performance rating, an aesthetic rating, a claustrophobia rating, a price rating, a value rating, a noise rating, a community popularity rating, a user interface size, a user interface weight, and a power savings rating.

64-129. (canceled)
